(19) United States
(12) Patent Application Publication
(10) Pub. No.: US 2013/0143244 A1
(43) Pub. Date: Jun. 6, 2013

(54) SOX9 AS A MARKER FOR AGGRESSIVE CANCER

(71) Applicant: Geetika Chakravarty, New Orleans, LA (US)

(72) Inventor: Geetika Chakravarty, New Orleans, LA (US)

(73) Assignee: ADMINISTRATORS OF THE TULANE EDUCATIONAL FUND, New Orleans, LA (US)

(21) Appl. No.: 13/817,431

(22) PCT Filed: Aug. 19, 2011
(86) PCT No.: PCT/US11/48481

§ 371 Date: Feb. 15, 2013

Related U.S. Application Data
(60) Provisional application No. 61/401,843, filed on Aug. 20, 2010.

(51) Int. Cl. G01N 33/68

(52) U.S. Cl. CPC G01N 33/6893
USPC 435/7.92; 435/7.1

(57) ABSTRACT

The invention provides methods for determining whether a cancer is or is likely to become aggressive, by detecting the presence of the transcription factor SOX9 in the cytoplasm of cells of the cancer, provided the cancer is not solid pseudopapillary tumor or a melanoma.

### CROSS-REFERENCE TO RELATED APPLICATIONS

This application claims the benefit of U.S. Provisional Application No. 61/401,843, filed Aug. 20, 2010, the contents of which are incorporated herein by reference.

### STATEMENT REGARDING FEDERALLY SPONSORED RESEARCH OR DEVELOPMENT

Not applicable.

### PARTIES TO JOINT RESEARCH AGREEMENT

Not applicable.

### REFERENCE TO SEQUENCE LISTING OR TABLE SUBMITTED ON COMPACT DISC AND INCORPORATION-BY-REFERENCE OF THE MATERIAL

Not applicable.

### BACKGROUND OF THE INVENTION

Invasive ductal carcinoma (“IDC”), the most common type of breast tumor, accounts for ˜70% of all reported cases of breast cancer in women and ˜80% of invasive breast cancers overall. IDCs are histologically diverse and show little consistency in tissue expression of common biomarkers such as Her2 neu and progesterone (PR) or estrogen receptors (ER). IDCs have the poorest prognosis of all human breast tumor types and continue to lack specific diagnostic tests that can potentially guide selection of patient-specific treatment regimen.

IDCs arise from non-invasive tumor tissue and rapidly spread to the lymphatic system and other surrounding tissues, suggesting that genes involved in orchestrating the distinctive interactions between the tumor cells and the surrounding extracellular matrix (ECM) may play a significant role in tumor progression. Indeed, gene expression profiling studies have identified characteristic signature genes that can predict clinical outcome of poor prognosis patients in retrospective studies. However, such profiling studies are primarily transcriptional readouts, and may not mimic protein-protein interactions that drive signaling pathways promoting metastatic growth. Hence, despite the development of sophisticated molecular profiling techniques, histological and genomic heterogeneity among cases of IDC continues to complicate the rational development of effective treatment strategies.

Recent studies have indicated that cancer cell invasiveness may directly be linked to epithelial mesenchymal transition (EMT), a process that is highly influenced by the host microenvironment. However, matrix remodeling features of tumor cells may not only depend on the action of stromal fibroblasts or diffusible factors in the tumor microenvironment, but also on the differentiation status of the carcinoma cell itself. If the carcinoma cells possess stem-like features, gene expression profiles can switch to that of a bone cell or an endothelial cell or undergo EMT to extravasate and intravasate target tissues to form micrometastasis. Thus, factors that govern stemness and EMT through their interactions with microenvironment-specific factors such as transforming growth factor-β (TGF-β), epidermal growth factor (EGF) and Wingless and integration site growth factor (Wnts), may represent promising targets for therapeutic intervention of invasive breast cancer. Indeed, these pathways have been shown to play a critical role in breast cancer metastasis. Some recent studies have reported that Twist and Foxc2 transcription factors play important role in luminal and basal breast cancer cell metastasis respectively. However, since only 40-50% of human tumors express these markers, it is likely that additional transcription factors may be involved in the progression of the remaining 50%-60% invasive breast cancer.

SOX families of genes encode cellular proteins that function as transcription factors. Upon stimulation, these genes initiate diverse signaling pathways that lead to regulation of cell growth, differentiation and lineage commitment. In addition to providing cardinal signals for embryonic development, certain SOX family members are also implicated in tumorigenesis. For example, overexpression of the pluoripotent stem cell marker SOX2 promotes proliferation and G1/S transition of breast cancer cells. In contrast, loss of human microRNA-126 and miR-335 have been shown to enhance breast cancer metastasis by targeting SOX4.

SOX9 is a HMG box transcription factor required for development, differentiation, lineage commitment and EMT during embryonic development. Studies involving hormone refractory prostate tumors, colorectal cancer and melanomas suggest that SOX9 may have a direct role in tumor growth. Interestingly, embryonic expression of SOX9 was observed in E14.5 and E17.5 mouse embryonic mammary bud, and has been reported in the mammary primordium of marsupials. It is also expressed in many human breast cancer cell lines, where its expression is induced in response to retinoic acid treatment and is regulated by Wnts in the intestinal crypts, hair bulge and the cartilage.

### BRIEF SUMMARY OF THE INVENTION

The present invention provides methods of determining whether a cancer is or is likely to be aggressive. In one group of embodiments, the invention provides methods of screening cancer cells in a sample for aggressiveness. The methods comprise detecting the presence or absence of cytoplasmic SOX9 in the cancer cells, wherein the presence of cytoplasmic SOX9 is an indication the cancer is more aggressive and the absence of cytoplamic SOX9 is an indication the cancer is less aggressive, provided the cancer cells are not from a solid pseudopapillary tumor or a melanoma. In some embodiments, the sample is from a human patient. In some embodiments, the detection of cytoplasmic SOX9 is by immunohistochemistry. In some embodiments, the detection of cytoplasmic SOX9 is by immunofluorescence. In some embodiments, the detection of cytoplasmic SOX9 is by western blotting. In some embodiments, the detection of cytoplasmic SOX9 is by enzyme-linked immunosorbent assay. In some embodiments, the cancer cells are breast cancer cells. In some embodiments, the breast cancer cells are ductal carcinoma cells. In some embodiments, the breast cancer cells are not invasive lobular carcinoma cells. In some embodiments, the cancer cells are head and neck cancer cells. In some embodiments, the head and neck cancer cells are squamous cell carcinoma cells.

In another group of embodiments, the invention provides methods of screening for aggressiveness cancer cells having cytoplasm and a nucleus. The methods comprise testing both the cytoplasm and the nucleus of said cells for the presence of SOX9, wherein the presence of SOX9 in the cytoplasm of the cells but not in the nucleus of the cells, or the presence of SOX9 in the cytoplasm of the cells in a quantity greater than SOX9 is present in the nucleus of the cells, is indicative of greater aggressiveness and the absence of SOX9 in the cytoplasm of the cells is indicative of lower aggressiveness, provided the cancer cells are not from a solid pseudopapillary tumor or a melanoma. In some embodiments, the cancer cells are from a human patient sample. In some embodiments, the cancer cells are breast cancer cells. In some embodiments, the breast cancer cells are ductal carcinoma cells. In some embodiments, the breast cancer cells are not invasive lobular carcinoma cells. In some embodiments, the cancer cells are prostate cancer cells. In some embodiments, the cancer cells are head and neck cancer cells. In some embodiments, the head and neck cancer cells are squamous cell carcinoma cells.

In yet another group of embodiments, the invention provides methods of screening cancer cells in a sample for higher or lower aggressiveness. The methods comprise visualizing the presence of SOX9 in the cytoplasm of the cells, wherein visualizing SOX9 in the cytoplasm of the cells but not in the nucleus of the cells is indicative of higher aggressiveness and the absence of SOX9 in the nucleus of the cells is indicative of lower aggressiveness, provided the cancer cells are not from a solid pseudopapillary tumor or a melanoma. In some embodiments, the sample is from a human patient. In some embodiments, the cancer cells are breast cancer cells. In some embodiments, the breast cancer cells are ductal carcinoma cells. In some embodiments, the breast cancer cells are not invasive lobular carcinoma cells. In some embodiments, the cancer cells are prostate cancer cells. In some embodiments, the cancer cells are head and neck cancer cells. In some embodiments, the In some embodiments, the head and neck cancer cells are squamous cell carcinoma cells. In some embodiments, the visualization is by immunohistochemistry. In some embodiments, the visualization is by immunofluorescence.

### BRIEF DESCRIPTION OF THE DRAWINGS

FIGS. 1A-C. FIG. 1A shows “box-and-whiskers” plots for SOX9 expression in three different sets of data. In each of panels (a), (b) and (c) within FIG. 1A, the plot on the left presents the data for ER− cells while the plot on the right plots the data for ER+ cells. FIG. 1B. FIG. 1B presents data for three different grades of breast cancer using breast cancer gene expression data-sets from studies as labeled. In each of the panels within FIG. 1B, the plot on the left presents data from grade I breast cancer, the center plot presents data from grade II breast cancers, and the plot on the right presents data from grade III breast cancers (labeled as “GR1,” “GR2,” and “GR3,” respectively). P values were generated using Student's t-test through Oncomine. For ease of referencing, the total numbers of samples analyzed in each data-set are listed below the box plots of the respective groups. As the platforms and cut-offs used for analysis varied between the original studies, results are presented separately for each study as per the clinicopathological variables analyzed. FIG. 1C. FIG. 1C is a Kaplan-Meier plot for the top and bottom 10% SOX9 expressors and association with patient survival using data obtained from van de Vijver, et al., N Engl J Med, 347:1999-2009 (2002). ER=estrogen receptor.

FIGS. 2A-B. FIG. 2A presents box plots showing that SOX9 expression intensities across all breast cancer versus normal breast tissue. FIG. 2B presents box plots of gene expression intensities in ductal versus lobular carcinomas. For both Figures: the statistical significance of differential SOX9 expression in cancer and normal and in ductal and lobular was determined using one-tailed Wilcoxson's rank sum test and two-tailed Mann-Whitney U tests (P≦0.0001 and P≦0.0001), respectively. Numbers in brackets indicate the total number of specimens analyzed in each category.

FIGS. 3A-B. FIGS. 3A and 3B present SOX9 and Ki-67 immunohistochemical (IHC) score distributions. FIG. 3A presents the IHC score distribution in 20 ductal carcinoma in situ (DCIS) specimens, while FIG. 3B presents the IHC score distribution in 66 invasive ductal carcinoma (IDC) specimens of breast cancer tissue microarrays. For both Figures, the X-axis shows the IHC scores of the specimens based on percentage of cells immunopositive for SOX9 or Ki-67. An IHC score of 0 implies no staining, a score of 1 implies staining of 0-10% cells; a score of 2 indicates 10-50% cells are immunopositive, and a score of 3 indicates >50% cells are immunopositive. For both Figures, the Y-axis shows the percentage of cases with different IHC scores for Ki-67 () or SOX9 (▪).

FIG. 4. FIG. 4 is a bar graph presenting the quantitation of cells with cytoplasmic or nuclear SOX9 localization. The data represents the mean±SEM from three random fields from three separate coverslips. The vertical axis presents the percentage of cells showing nuclear or cytoplasmic SOX9 per total number of cells. The horizontal axis shows the results for four different cell lines. For each of the cell lines, the left hand bar shows the percentage of cells with nuclear SOX9 and the right hand bar shows the percentage of cells with cytoplasmic SOX9.

FIGS. 5A-C. FIG. 5A. FIG. 5A is a bar graph showing cytoplasmic SOX9 containing MDA-MB-231 cells show negligible activation of the Col2a1 reporter (grey bar) as compared to cells transfected with vector alone (white bar). However, transfection of wild type SOX9 in these cells results in six fold higher activation of the col2a1 reporter (black bars) as compared to cells with endogenous SOX9 (Grey bars). FIG. 5B. FIG. 5B is a bar graph showing that cells with cytoplasmic (“cytop”) SOX9 show 16 fold higher activation of the TOP-flash reporter in wnt3a treated cells (black bar) as compared to untreated cells (white bar). Once again, transfection of wild type SOX9 in these cells results in much lower induction (22 fold as opposed to 127 fold in cells with cytoplasmic SOX9) of the TOP-flash reporter in the wnt3a treated cells as compared to the untreated cells. Data represents Mean±SEM from two independent sets of experiments done in triplicates. The wild type SOX9 was DDK tagged and could be distinguished from the endogenous SOX9 protein using a mouse monoclonal anti-DDK antibody. This antibody localized the wild type SOX9 in the nucleus. Nuclei were counter stained with DAPI, while wild type SOX9 expressing cells were visualized with Alexa 594 secondary antibody and merged images showed the transfected SOX9 was localized in the nucleus. FIG. 5C. FIG. 5C is a bar graph showing the effect of SOX9 localization on cell proliferation of two cell lines, as assessed by the MTT assay. Proliferation of MCF7 cells, which have mostly nuclear expression of SOX9, was significantly inhibited (two tailed t-test p=0.026) when the serum starved cells were grown in the presence of 10% serum as compared to cells that continued to grow in 0.5% serum media. In contrast, serum starved MDA-MB-231 cells, which have cytoplasmic expression of SOX9, continued to proliferate whether they were grown in 0.5% serum media or 10% FBS media (two tailed t-test p=0.7554). Data represents Mean±SEM from three independent sets of experiments

FIGS. 6A-B. FIG. 6A. FIG. 6A presents four representative FACS plots analyzing cell cycle of Serum free (left field) or 10% FBS treated (right field) MDA-MB-231 (top field) or MCF-7 cells (bottom field). Note almost a two fold increase in the S phase fraction of MDA-MB-231 cells when they were grown in SFM versus 10% FBS media as opposed to 1.3 fold increase in MCF7 cells. In contrast, a much higher percentage of MCF7 cells were arrested in G2M phase of the cell cycle compared to a negligible change in the G2M fraction of MDA-MB-231 cells with or without 10% FBS. FIG. 6B. FIG. 6B is a bar graph showing the proportion of cells in G0, G1, S and G2/M and apoptotic phases of cycling MDA-MB-231 and MCF7 cells grown with or without 10% FBS. Values presented are Means±SEM derived from two independent experiments.

FIG. 7. FIG. 7 sets forth four graphs of growth (%) plots demonstrating the effect of increasing concentration of TSA and LMB on the growth of MDA-MB-231 cells. Cells were exposed to the respective drugs (as shown on the horizontal axis) at the indicated concentrations for 48 h and cell viability was measured by the MTT assay. Results were normalized to those of the vehicle-treated cells and reported as growth relative to control. Data presented are the means of three independent experiments±SEM. Each treatment was done in replicates of eight. The results show TSA exposure confers markedly heightened sensitivity to growth inhibition in vitro (left graph, top field). However, MDA-MB-231 cell growth is unaffected in response to LMB treatment (left graph, bottom field). The graphs on the right side show the log transformed curves of data presented in the graphs on the left side.

FIGS. 8 A-B. FIG. 8A. FIG. 8A is a photo of western blots showing the effects of TSA (500 nM) or LMB (5 ng/ml) treatment for 4 h on MDA-MB-231 cell total protein and acetylated SOX9 expression. Control MDA-MB-231 cells (lane 1) were grown in SFM, treated for 4 h with vehicle or grown in regular 10% FBS media (lane 2) or 5 ng/ml LMB (lane 3) or 500 nM TSA (lane 4) and then western-blotted using antibodies to the proteins as set forth in Example 4. FIG. 8B. FIG. 8B panels (a) and (b) show the effects of SOX9 knockdown on its protein level and growth of MDA-MB-231 clones. Panel (a): MDA-MB-231 cells transduced with shRNA for SOX9 show downregulation of the protein in clone II and IV but its levels are unaffected in cells transduced with the non silencing shRNA. Panel (b): 200 μl of 1×10⁴ cells/ml of MDA-MB-231 non silencing shRNA clone (hollow bars) and two SOX9 shRNA clones (Clone II: dotted and Clone IV: vertical striped bars) were grown overnight in SFM and left untreated (white bars) or treated with either 0.5% FBS (light grey bars) or 10% FBS (steel grey bars). After another 48 h of culture, percentage proliferation was measured as detailed in Example 4. Both SOX9 shRNA clones II and IV had significantly higher proliferation rate (two tailed t-test p<0.0001) as compared to the non silencing shRNA clone when grown in 10% serum after 24 h of serum deprivation. SEs are based on triplicate set of experiments and each treatment was tested in replicates of eight.

### DETAILED DESCRIPTION

### Introduction and Overview

Surprisingly, it has been discovered that the transcription factor SOX9 is a marker that can be used as a prognostic indicator for the progression of cancers in human subjects. In particular, the studies underlying the present invention indicate that the expression and localization of SOX9 changes as cancers evolve and that identifying where SOX9 is compartmentalized in cancer cells can serve as a prognostic marker reflecting the inherent aggressiveness of those cancer cells. Using breast cancer as an exemplar, the studies underlying the invention revealed that SOX9 expression was virtually undetectable in normal breast tissue, was expressed in the nucleus in benign lesions and non-invasive forms of breast cancer, and was expressed in the cytoplasm, but not in the nucleus, in invasive, metastatic cancers. Further, more than 60% of the cancers lacking estrogen receptors showed the presence of cytoplasmic SOX9. The loss of estrogen receptors in breast cancers is indicative that the cancer is no longer sensitive to hormonal therapy and needs to be treated with more aggressive interventions. Thus, the studies underlying the invention revealed not only that the expression pattern of SOX9 is different in cancers with different levels of metastatic, or invasive, potential, but also that the detection of cytoplasmic SOX9 in cancer cells is an indication the cancer is or is becoming more aggressive than that of cancers that do not express detectable cytoplasmic SOX9. For example, less invasive breast cancers are often estrogen receptor positive and therefore can be treated with hormonal therapies that block the action of estrogen on the cancer cells or that inhibit the production of estrogen. The presence of cytoplasmic SOX9 in breast cancer cells taken from a patient being treated with hormonal therapy, however, would indicate that the cancer is becoming or is likely to become more invasive and that the clinician should consider changing the patient's treatment to a more aggressive intervention, such as chemotherapy, radiation or surgery. A finding that cells of a breast cancer have cytoplasmic SOX9 and is ER− would likewise indicate that the patient has a cancer that is or is likely to become invasive and should be treated with more aggressive therapies, such as chemotherapeutics.

As noted, SOX9 is a transcription factor. Its function seems in part to be cell cycle arrest. To Transcription, of course, occurs in the nucleus and to be functional, SOX9 must translocate from the cytoplasm, where it is synthesized, to the nucleus. Without wishing to be bound by theory, it is believed that the presence of SOX9 in the cytoplasm but not in the nucleus of more aggressive cancers may be due to interference with the translocation of SOX9 from the cytoplasm to the nucleus, that this interference prevents SOX9 from performing its normal role in controlling cell proliferation, and that this interference is therefore in some part responsible for the cancer's progression to a more invasive phenotype.

The findings reported herein with respect to breast cancer led to the thought that similar expression patterns would be found in other cancers that share features with breast cancer, such as having a morphogenesis involving formation of ducts and lobules, regulation through endocrine mechanisms, such as thyroid cancer, and origination from tissues that utilize regular self renewal to maintain tissue homeostasis, such as oral squamous cell carcinoma.

To determine if these considerations were valid, tumor samples from head and neck cancers were examined for expression of SOX9. Samples of head and neck cancers that were highly metastatic were found to have much higher expression of cytoplasmic SOX9 than samples from normal tissues. In particular, cytoplasmic SOX9 was several fold higher in highly metastatic oral squamous cell carcinomas and metastatic anaplastic and follicular thyroid carcinoma cell lines than in normal oral epithelium or normal thyroid cells. This second example of SOX9 localization being correlated with aggressive tumors confirms the prediction that the presence of cytoplasmic SOX9 can be used as a general predictor that the cancer is or is likely to become aggressive. Patients whose cancers have cytoplasmic SOX9 should therefore be treated with aggressive interventions, such as chemotherapy, surgery, radiation, biologics, or a combination thereof. The invention therefore provides an important new source of information to help oncologists and other practitioners determine whether and when to institute aggressive therapeutic measures.

In general, the studies noted to date regarding the presence of SOX9 in cancer cells have reported detecting SOX9 in the nucleus. Cytoplasmic SOX9 has been reported in solid pseudopapillary tumor, a rare pancreatic cancer usually found in children (Galmiche et al., Histopathology, 53 (3):318-24 (2008)) and in some melanoma cells in a skin model in which melanocytes were replaced with melanoma cells (Passeron et al., J Clin Invest, 119 (4):954-963 (2009)). Solid pseudopapillary tumors and melanomas are excluded from the cancers encompassed by the inventive methods.

While not all aggressive cancers are expected to have SOX9 in the cytoplasm of their cells, the studies herein indicate that cancer cells which do have cytoplasmic SOX9 are either aggressive or likely to become aggressive and to have a poorer prognosis. Detection of the presence of cytoplasmic SOX9 in cells from a patient's cancer therefore indicates that the practitioner should consider a treatment plan or regimen for the patient that is appropriate for an aggressive cancer.

In some embodiments, the cancer is a breast cancer, a head and neck cancer, a prostate cancer (other than a solid pseudopapillary tumor), or an ovarian cancer. In some more preferred embodiments, the cancer is a breast cancer or a head and neck cancer. In some of these embodiments, the head and neck cancer is a squamous cell carcinoma or thyroid cancer. In some preferred embodiments, the cancer is an anaplastic or follicular thyroid carcinoma. In some embodiments in which the cancer is a breast cancer, the breast cancer is invasive ductal carcinoma. In some embodiments, the breast cancer is not a matrix-producing carcinoma of the breast. In some embodiments, the breast cancer is not an invasive lobular carcinoma. In some embodiments, the cancer is not a colorectal cancer. In some embodiments, the cancer is not a lung adenocarcinoma.

### DEFINITIONS

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Accordingly, the terms defined immediately below are more fully defined by reference to the specification in its entirety.

As used herein, “SOX-9” and “SOX9” refer to a 509 amino acid human transcription factor whose sequence is set forth in the National Center for Biotechnology Information's Protein Database under accession no. NP_{—}000337.

“ER” stands for “estrogen receptor.” “ER+” refers to breast cancer cells that are positive for estrogen receptors, and therefore are responsive to hormonal therapy. “ER−” refers to breast cancer cells that are negative for estrogen receptor, and therefore are no longer responsive to hormonal therapy.

As used herein, “aggressive” refers to a cancer that is likely to proliferate, to be invasive, or both, and which are therefore associated with a poorer prognosis.

“Solid pseudopapillary tumor” is a rare pancreatic tumor, particularly found in children. Galmiche et al. reported in 2008 that 7 of 8 such tumors examined had strong cytoplasmic expression of SOX9, but no nuclear expression. Galmiche et al., Histopathology, 53 (3):318-24 (2008).

### SOX9

SOX genes are a family of genes encoding transcription factors with a highly conserved “high mobility group” (HMG) DNA binding domain. There are 20 SOX genes in humans, which are expressed during development. See generally, Thomsen et al., Differentiation, 76:728-735 (2008).

SOX9 is a 509 amino acid human transcription factor whose sequence is set forth in the NCBI Protein database under accession no. Accession No. NP_{—}000337. Discovered in the 1990's, it was implicated early on in embryonic cartilage development, sex differentiation, pre-B and T cell development and neural induction. Pevny and Lovell-Badge, Curr Opin Genet Dev, 7:338-344 (1997). It was later found to be important for the development of numerous organs and tissues, including the pancreas, the prostate, the intestines, and pigment cells. Like other genes important in development, its deregulation or dysregulation has been associated with some cancers, including pancreatic cancer, prostate cancer, colorectal cancer, cutaneous basal cell carcinoma, melanoma, and mesenchymal chondrosarcoma. Jiang et al. (Clin Cancer Res 16:4363-4373 (2010)) reported that they found SOX9 expression to be upregulated in human lung cancer.

### Breast Cancer

As the name implies, breast cancers are cancers that start in tissues of the breast. The two main forms are ductal carcinoma, originating in the breast ducts, and lobular carcinoma, originating in the lobules. Each of these has a noninvasive form called in situ. Ductal carcinoma in situ, or “DCIS”, is a breast cancer of the ducts that has not invaded other tissue. Breast cancers expressing estrogen receptors on their surface, referred to as ER+, are responsive to estrogen. Treatment of ER+ cancers often involves hormonal therapy, which may include tamoxifen to block the effect of estrogen or an aromatase inhibitor, which blocks estrogen production. Cancers progressing to more invasive or metastatic forms often no longer express estrogen receptors, and are thus referred to as “estrogen receptor negative”, or “ER−”. Treatment of ER− breast cancers cannot be done by hormonal therapy and may involve surgery, chemotherapy, biologics, radiation, or some combination of these. In preferred embodiments, the presence or absence of estrogen receptors on the breast cancer cells is correlated with the presence or absence of cytoplasmic SOX9. The presence of cytoplasmic SOX9, particularly in ER− cells, is strongly indicative of a poor prognosis and indicates the oncologist or other clinician should consider treating the patient with chemotherapeutics or other aggressive interventions.

### Methods of Detecting Cytoplasmic SOX9

The presence of cytoplasmic SOX9 can be detected using any of a number of conventional techniques. The studies underlying the invention detected the presence of SOX9 in the nucleus and cytoplasm of cells using immunohistochemistry (IHC). IHC is particularly useful for visualizing (seeing) whether SOX9 is present in the nucleus, in the cytoplasm or in both. IHC is well known in the art, and it is expected that persons of skill are familiar with the techniques of tissue collection, fixation, and sectioning used in protocols for sample preparation for IHC. It is further expected that the artisan is familiar with various enzymes and other reporter molecules that can be conjugated or fused to an anti-SOX9 antibody for visualization of the antibody-SOX9 interaction. The particular SOX9 antibodies and IHC techniques used in the studies underlying the invention are discussed in detail in the Examples.

While IHC is a particularly preferred technique for detecting the presence of cytoplasmic SOX9, other conventional techniques such as immunocytochemisty or immunofluoresence can be used. Immunofluorescence, immunoblotting, and other techniques used in the studies underlying the invention are described in the Examples. As with IHC, it is expected that persons of skill are familiar with all of these techniques and, indeed, immunocytochemistry is similar to IHC but is carried out on cells rather than tissue. While the fixation and antigen retrieval steps therefore may differ, the ability to determine the presence and compartmentalization of cytoplasmic SOX9 is therefore much the same.

In other embodiments, cytoplasmic SOX9 and nuclear SOX9 can also be detected by fractionating cell samples by conventional techniques to isolate the cytoplasmic portion and, if desired, the nuclear fraction. Typically, isolating the desired cellular fraction involves lysing the cell or cells of interest and centrifuging the lysate to obtain the fraction of choice. Reagents and kits for fractionating and obtaining cytoplasmic and nuclear proteins such as SOX9 are commercially available. While it is expected that persons of skill are familiar with techniques and materials for extracting cytoplasmic and nuclear proteins, the following is mentioned for the reader's convenience. The NE-PER Nuclear and Cytoplasmic Extraction Reagent Kit (Thermo Fisher Scientific, Rockford, Ill., catalog no. 78833) is stated by the manufacturer to be a reagent-based protocol that enables the stepwise lysis of cells, separation of the cytoplasm from the intact nuclease and then extraction of nuclear proteins away from genomic DNA and mRNA. The manufacturer states that both active nuclear proteins and cytoplasmic proteins can be recovered from the same cell culture or tissue sample. Another commercially available kit for fractionating cells, in this case adherent cells, is the Cell Fractionation Kit-HT, catalog no. MS862 (MitoSciences®, Eugene, Oreg.). The kit is based on sequential detergent extraction of cytosolic, mitochondrial and nuclear proteins without the need for mechanical disruption of cells. According to the manufacturer, the kit allows the measurement of any proteins which are differentially represented in the cytosol, mitochondria and nuclei, and is particularly applicable to studies of proteins that translocate between these three cellular compartments. It can thus be used for the study of the compartmentalization of SOX9, which translocates between the cytoplasm and the nucleus. The Nuclear and Cytoplasmic Extraction Kit, catalog no. 786-182 (G-Biosciences, Maryland Hts., MO) permits the clean separation of cytoplasmic proteins from nuclear proteins. Finally, the NC Protein extraction kit, catalog no. N2100-050 is available from BIOTANG Inc. (Waltham, Mass.).

Once the cytoplasmic protein fraction is obtained, the presence of SOX9 in the fraction can be determined by art-standard analytical techniques, such as by western blot or enzyme-linked immunosorbent assay (ELISA). In some embodiments, the nuclear fraction is also analyzed for the presence of SOX9 by the same technique(s) and, in some embodiments the quantity of SOX9 in the respective compartments (cytoplasm vs. nuclear) may be determined so they can be compared.

Monoclonal and polyclonal anti-SOX9 antibodies that can be used to detect SOX9 in the cytoplasm of human cancer cells, such as human breast cancer cells, are commercially available from several sources. Among the companies selling such antibodies are: Chemicon (available from Millipore, Billerica, Mass., catalog no. AB5535); Abcam®, (Cambridge, Mass.), which sells 12 anti-SOX9 antibodies (“Abs”), including a mouse monoclonal Ab, catalog no. ab76997, which the manufacturer states is suitable for IHC using paraffin embedded sections, western blotting, ELISA, and immunofluorescence, and a rabbit polyclonal Ab, catalog no. ab71762, stated to be suited for use in IHC using paraffin embedded sections, western blotting, and ELISA; and Abnova Corp. (Walnut, Calif.), which sells a number of anti-SOX9 monoclonal and polyclonal antibodies, including a mouse monoclonal antibody, catalog no. H00006662-M02, which the manufacturer states is useful for IHC using formalin-fixed, paraffin embedded sections, western blotting, ELISA, and immunofluorescence; and a rabbit polyclonal anti-SOX9 antibody Abnova states is useful for western blotting, IHC, and ELISA.

### EXAMPLES

### Example 1

This Example sets forth the materials and methods used in the studies reported in Examples 2 and 3, below.

Differential expression of SOX9 with respect to estrogen receptor status and grade was computed from datasets available from Oncomine 4.4 Research Edition (Compendia Bioscience, Inc., Ann Arbor, Mich.). Oncomine's gene search function was used to locate microarray studies for which gene expression data were publicly available. Studies were further queried to determine if they also enlisted information on prognostic indicators of breast cancer such as histological grade and ER status in addition to the expression unit data for SOX9 in breast cancer. Data obtained for individual studies was processed and normalized by Oncomine and used directly for differential expression analysis of SOX9. Results were sorted based on each class of analysis and used to create boxplots. Meta analysis of these studies was not performed as some of these studies used different array platforms for hybridization and could not be combined. Data for the survival analysis was from the Integrated Tumor Transcriptome Array and Clinical data Analysis (“ITTACA”) database (Institute Curie, Paris, France) website, which can be accessed by entering the following terms together in a web browser as one search string: “bioinfo-out.”, “curie.fr”, “/ittaca/” (the terms are separated here to avoid creation of an active hyperlink when this text appears on-line).

Breast tumor Tissue Microarrays (TMA) containing 206 cores of grade I-III breast tumors was purchased from Tissue Array Network (Rockville, Md.). Specifically, the array contained 152 cores of breast carcinoma [32—lymph node metastasis, 68—invasive ductal (IDC), 22—invasive lobular (ILC), 22—intraductal (DCIS), 4—lobular carcinoma in situ (LCIS) and 4—squamous cell carcinoma (SCCA)]. There were additional 40 cases of benign breast tissue that included 10 samples of adjacent to tumor normal breast parenchyma, 6 normal tissues, 16 hyperplasias, 16 inflamation and 8 fibroadenoma.

Antibodies:

The following primary and secondary antibodies were used at the specified dilutions: anti-SOX9 (1:250) from Chemicon (a unit of Millipore, Billerica, Mass.); anti-Ki-67 (1:200) from Biocare Medical (Concord, Calif.); streptavidin horseradish peroxidase and biotinylated goat antirabbit IgG from Dako (Dako North America, Inc., Carpinteria, Calif.).

Controls:

Human adult skin sections were included as positive controls for SOX9 protein expression by IHC. Non-specific staining (negative control) was obtained by pre-adsorbing the antibody with the peptide antigen used to raise the antibody. However, due to limited availability of the peptide antigen, additional negative control was obtained by omitting the primary antibody, and replacing it with normal rabbit serum from Dako.

Immunohistochemistry:

SOX9 and Ki-67 immunohistochemical detection was performed on paraffin embedded TMA with a rabbit polyclonal SOX9 and mouse monoclonal Ki-67 antibody using a DAKO autostainer in accordance with manufacturer's recommendations. CAT Hematoxylin was used to counter stain the specimens. Blind immunohistochemical scoring was performed by a pathologist, and the scoring was confirmed by two more “blind” observers. Signals were considered positive when brown staining was observed either in the cytoplasmic or nuclear compartment. Intensity was scored as 0 (no signal), + (weak=1), ++ (moderate=2), +++ (marked=3).

Plasmids and Transient Transfection of SOX9 cDNA:

Full length cDNA for SOX9 was cloned into pCMV6 vector. Transient transfection studies were performed using Fugene 6 (Roche Diagnostics, Indianapolis, Ind.) as per the recommendations of the manufacturer. Briefly, 293T cells were plated in 6 well tissues culture dishes containing glass cover slips at 60%-70% confluency. Cells were transfected with Fugene (2 μg of DNA and 6 μl of Fugene per well). Immunofluorescent detection was performed 24 h after transfection.

Statistical Methods:

Statistical significance of SOX9 expression was determined using non-parametric tests. A one-tailed Wilcoxson rank sum test was used for cancers vs. normal cases, while the two-tailed Mann Whitney test was used for the ductal vs. lobular ones. The two-sample unequal variance t test was used to determine the significance of differential expression of SOX9 in ER− vs. ER+ (raw normalized expression units) and in Grade I vs. Grade III. Kendall's tau test was used to determine the correlation between cytoplasmic SOX9 expression and the Ki-67 expression. Follow up data were measured from the date of diagnosis to the date of last news for live patients for overall survival and a plot of the Kaplan-Meier estimate of the surviving fraction was generated. The two groups were compared with log rank tests using the Graph Pad Prism 5 software (GraphPad Software, Inc., La Jolla, Calif.).

### Example 2

This Example reports the results of a first group of studies conducted in the course of the present invention.

SOX9 Expression is Significantly Associated with Estrogen Receptor Negative and Higher Grade Human Breast Tumors:

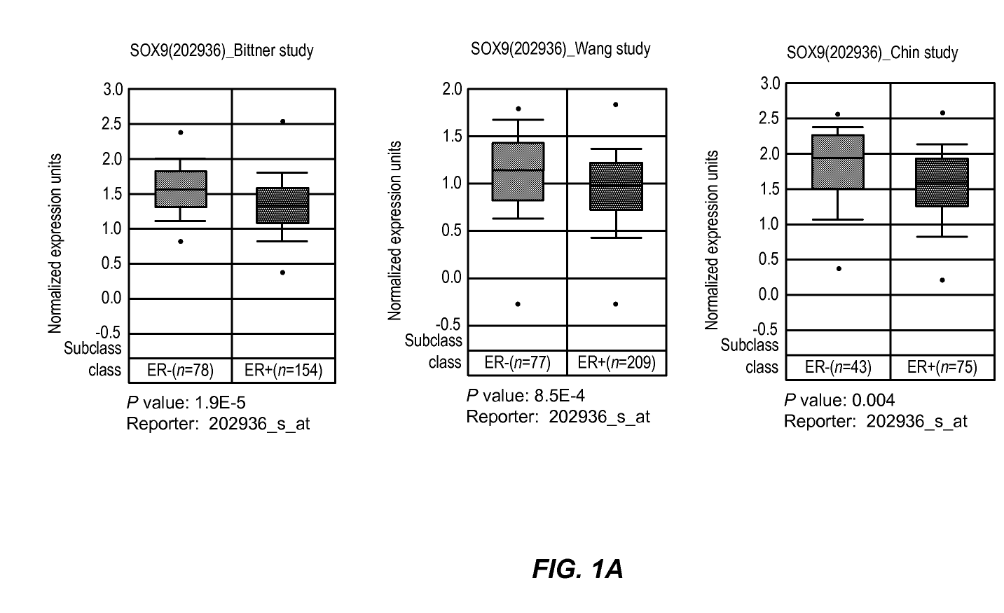

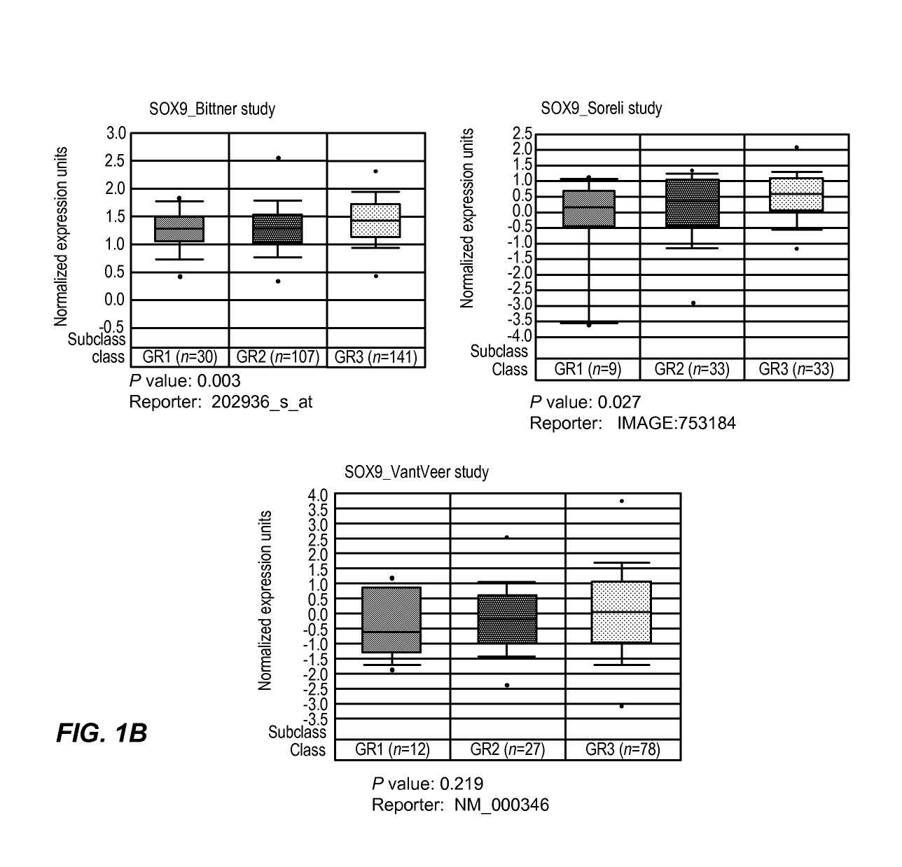

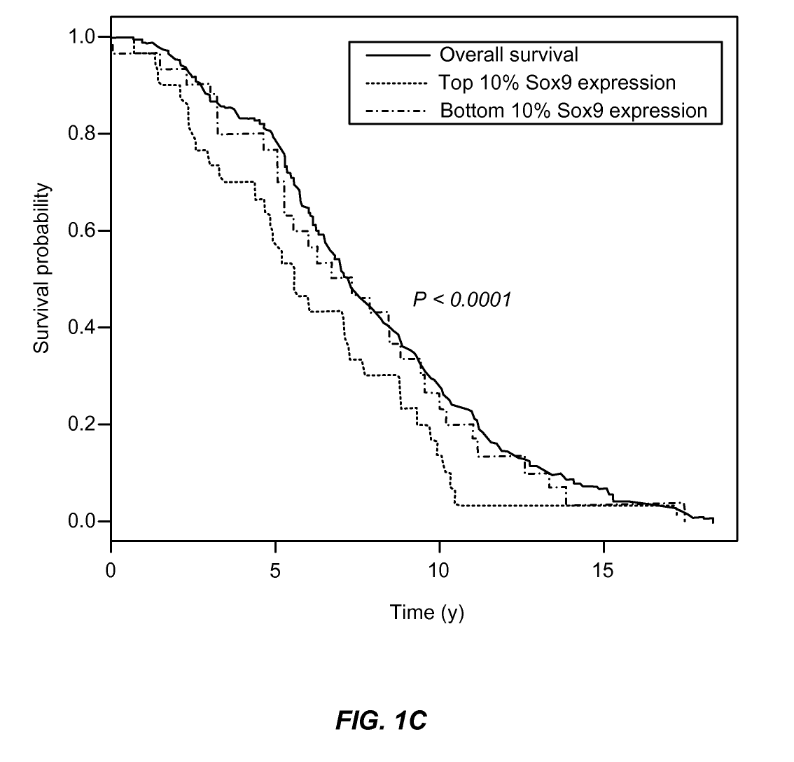

To investigate whether SOX9 was over expressed in human breast tumors and to determine its relationship with ER status, tumor specific SOX9 mRNA expression data were down loaded from the Oncomine or ITTACA websites and analyzed to look for differential expression of SOX9 with respect to ER status and histological grade. Higher SOX9 expression (as detected with the probe set 202936_s_at) was significantly associated with ER negative phenotype in three separate studies. Specifically, the mean SOX9 expression in ER+tumors in the Wang et al. (Lancet, 365(9460):671-9 (2005), Chin et al. (Cancer Cell, 10(6):529-41 (2006) and Bittner (NCBI's Expression Project for Oncology (“expO”) Gene Expression Omnibus (“GEO”) database, on-line on the NCBI GEO accession display under Series GSE2109) studies (FIG. 1A) was 8.12, 5.52 and 8.3 arbitrary units respectively, whereas it was 11.32, 7.6 & 16.94 units respectively in ER− tumors indicating a 40-100% increase in SOX9 expression in the ER negative group. Comparison of mean SOX9 expression units with t-tests in the two groups of tumors further confirmed that SOX9 was significantly over-expressed in ER− tumors compared to the ER+ tumors (p≦0.001) in all three of these studies (FIG. 1A). With respect to the analysis herein of Grade I, II, and III breast tumors, at least two out of the three studies found a significant increase in SOX9 expression with the increase in histological grades (p≦0.01, Gr I vs Gr III, FIG. 1B), even though all of these studies utilized different reporters (202936, 753184, NM_{—}000346) to monitor SOX9 expression.

Higher SOX9 Expression Correlates with Decreased Overall Survival:

SOX9 expression values downloaded from a public database of 259 patients with invasive breast cancers showed that over-expression of SOX9 also influenced a patient's overall survival. Although the dataset's top 10% SOX9 expressors and the bottom 10% SOX9 expressors shared the same characteristics until about 21 months, survival probability among the top 10% SOX9 over expressers began to drop considerably (FIG. 1C). More specifically, the 50% survival probability in the top 10% group was lowered by approximately two years in this data set, as can be seen by the fact that the bottom 10% group had a 50% survival probability of 7 yrs and 6 months as opposed to 5 yrs and 8 months in the top 10% group. Kaplan-Meier curve comparisons using the Log-rank (Mantel-Cox) test showed that this difference was statistically significant (p=0.0005).

SOX9 Protein Expression is Pronounced in Breast Cancers but Undetectable in Normal Breast Mammoplasty Tissue:

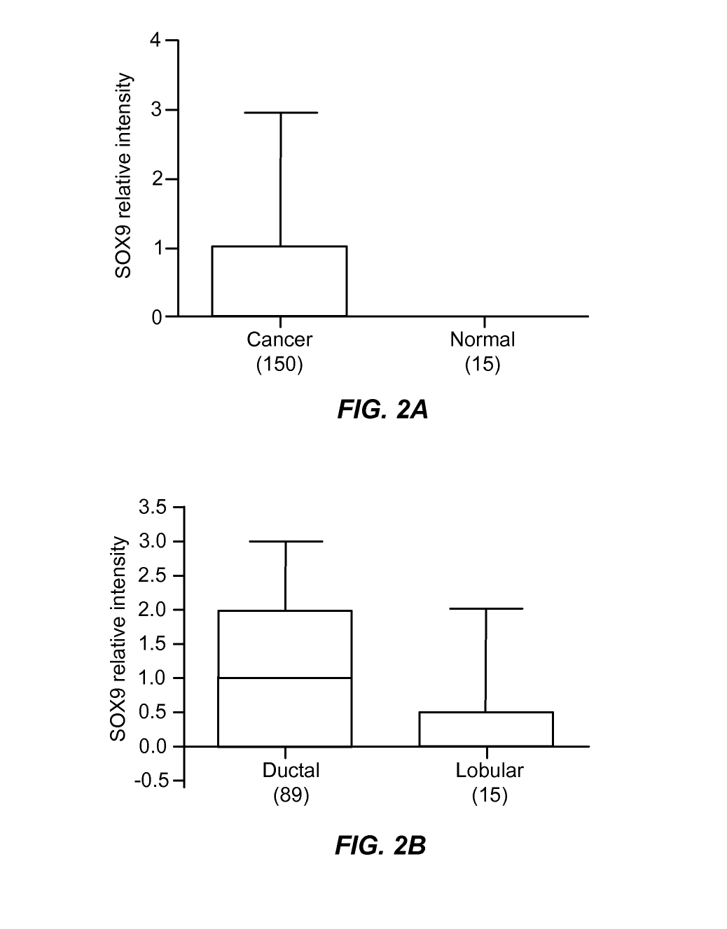

When a commercially available breast tumor TMA was studied for SOX9 protein expression using immunohistochemical (IHC) detection methods, SOX9 protein was detected in ductal epithelial cells of atypical ductal hyperplasia, DCIS, IDC and Lymph node metastasis samples (Table 1). With the exception of one sample that was classified as ductal ectasia, however, all other adjacent-to-tumor normal breast tissues and the six core biopsies containing normal breast mammoplasty samples were negative for SOX9 staining. Similarly, the relative intensity of SOX9 in carcinoma samples (n=150, two cores had no tissue) was significantly higher as compared to the normal breast specimens (n=15, includes NAT+ normal mammoplasty specimens; one core biopsy was lost in the staining process). In particular, the median intensity score for the carcinoma samples was 1, while it was zero for all the normal breast samples analyzed (FIG. 2A). This difference was highly significant as determined using a Wilcoxon signed rank test (p<0.0001). This was also true when ductal carcinoma samples (n=90, but one biopsy had no tissue) were compared with the NATs (n=9, one sample excluded from analysis because of low cellularity or complete loss).

When lobular carcinoma samples (ILC+LCIS, n=26) were compared with NAT parenchyma, however, this difference was lost as only 1 in 26 lobular carcinoma samples was positive for SOX9. By corollary, SOX9 expression was more pronounced in the ductal lineage specimens as compared to the lobular carcinoma in situ, and the invasive lobular carcinoma samples (P=0.008, Mann Whitney test U test; FIG. 2B). No staining was observed in the absence of the primary antibodies, or with non-specific immunoglobulin controls.

SOX9 expression is cytoplasmic in a subset of human breast carcinomas: In addition to the higher expression of SOX9 mRNA and protein in tumors, cytoplasmic expression of SOX9 was observed in DCIS (4/22, or 18%), IDC (18/68, or 26%) and lymph node metastases (3/32, or 10%) (Table 1). The two-tailed Fisher's exact test showed that when invasive specimens (IDC+LN) were compared with all other specimens including NAT & normal, cytoplasmic SOX9 expression significantly associated with invasive cancers (p=0.01). To ensure that the antibody was not cross reacting with non specific cytoplasmic proteins, HEK 293T cells were transfected with a full length SOX9 cDNA construct and immuno stained with SOX9 polyclonal antibody. Transfected HEK 293T cells exhibited strong nuclear staining with no staining in the non transfected cell nuclei or cytoplasm. Human adult skin sections used as positive controls for SOX9 staining and stained in parallel with the TMA, further substantiated this observation. Positive staining in the skin section with dermal nevus was confined to the nuclei of nevus cells and dermal melanocytes. The specificity of cytoplasmic localization of SOX9 in the advanced invasive tumors was also evident from the fact that an atypical ductal hyperplasia (ADH) exhibited positive immunostaining mainly in the nuclei of the epithelial cells. However, the nuclei of surrounding stromal cells or infiltrating inflammatory cells in the invasive carcinoma or the lymph node metastasis specimens failed to show any SOX9 staining, although, a very small percentage of tumors (1/68, or ≅1.5%) did exhibit both cytoplasmic and nuclear localization of SOX9.

TABLE 1
Immunohistochemical analysis summary of SOX9 expression in
human breast carcinomas and normal breast tissue.

| Sox9 | | | | | % showing |
|---|---|---|---|---|---|
| Expres- | | | Cytoplasmic + | Total | cytoplasmic |
| sion | Cytoplasmic | Nuclear | Nuclear | Cases | accumulation |
| LN Met | 3 | 0 | 0 | 32 | 3/32 (9%) |
| IDC | 18 | 3 | 1 | 68 | 18/68 (26%) |
| ILC | 0 | 1 | 0 | 22 | 0/22 (0%) |
| SCCA | 0 | 0 | 0 | 4 | 0/4 (0%) |
| DCIS | 4 | 0 | 0 | 22 | 4/22 (18%) |
| LCIS | 1 | 0 | 0 | 4 | 1/4 (25%) |
| NAT | 0 | 0 | 1* | 10 | 1/10 (10%) |
| Normal | 0 | 0 | 0 | 6 | 0/6 (0%) |

*/Indicates adjacent normal tissue with ductal ectasia
Legend to Table 1:
LN Met: lymph node metastasis;
IDC: invasive ductal carcinoma;
ILC: invasive lobular carcinoma;
SCCA: squamous cell carcinoma;
DCIS: ductal carcinoma in situ;
LCIS: lobular carcinoma in situ;
NAT: adjacent-to-tumor normal parenchyma.

Cytoplasmic SOX9 staining is significantly associated with proliferation marker Ki-67 staining. Based on the fact that SOX9 is known to be induced in response to growth arresting/differentiating signals such as retinoic acid, the question arose whether its cytoplasmic accumulation confers a proliferative advantage to a tumor cell. To test this hypothesis, a serial section of the TMA was immuno-stained with a proliferation marker, Ki-67, and immunoscored to determine if tumors exhibiting cytoplasmic accumulation of SOX9 stained for Ki-67 as well. In 204 paired specimens scored for both SOX9 and Ki-67 staining, the probability of SOX9 and Ki-67 staining occurring together was determined by calculating the conditional probability P(A|B)=P(A and B)/P(B) where A=“Ki-67>0” and B=“SOX9>0”. The data indicated that, in 54 out of 84 cases, Ki-67 staining was more likely in the event of SOX9 staining. This correlation was determined to be highly significant using the non-parametric Kendall's tau test (Kendall's tau=0.337 with a p-value <0.0001). This association of cytoplasmic accumulation of SOX9 with Ki-67 expression was more pronounced in the invasive ductal breast tumors. It is worth noting that nuclear staining of SOX9 in a lone invasive lobular carcinoma was not accompanied by increased Ki-67 staining.

Diversity in the Distribution of Immunohistochemical Scores for SOX9 and Ki-67 in DCIS:

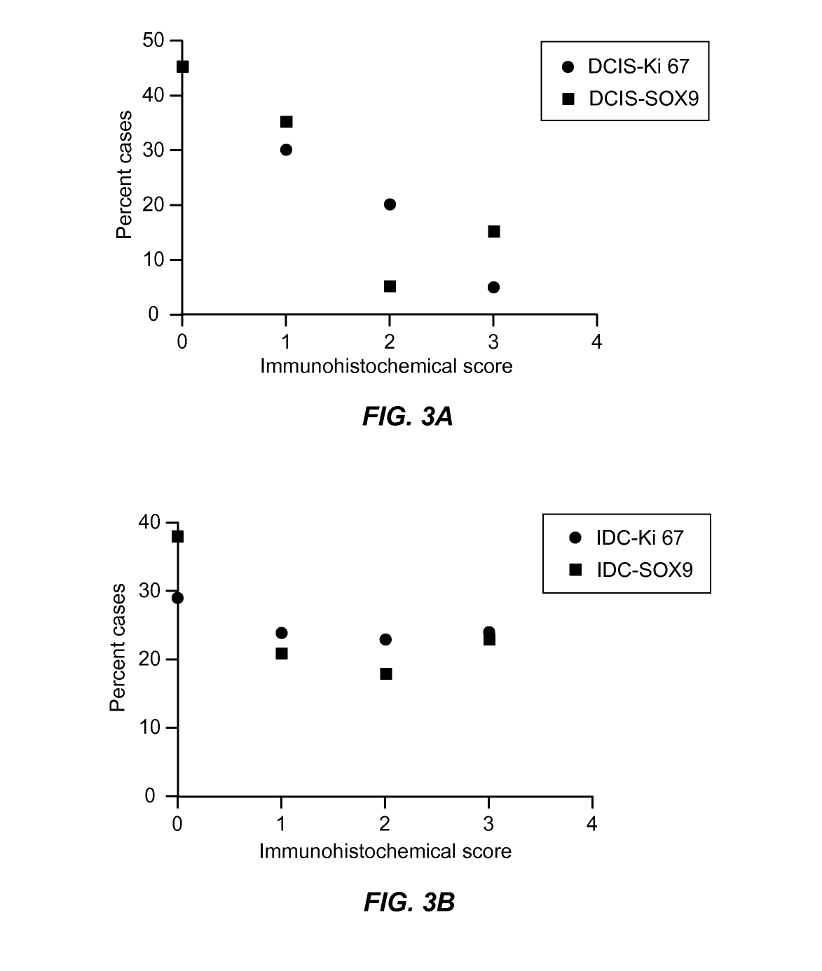

Intertumoral heterogeneity in the biological responsiveness of different breast tumors was apparent in the array from the SOX9 localization data in different stages of breast cancer, and also from the varied expression of Ki-67 in these specimens. Accordingly, the distribution of SOX9 and Ki-67 immunohistochemical (IHC) score in DCIS and IDC specimens were analyzed to compare the outcome with an earlier report (Allread et al., Clin Cancer Res 14:370-8 (2008) that had highlighted the importance of emergence of diversity during breast cancer evolution. Immunohistochemical scores of SOX9 and Ki-67 showed wide variability in their distribution amongst the IDC and DCIS specimens) FIGS. 3A and 3B). Specifically, DCIS specimens with a higher IHC score of 2 and 3 for SOX9 and Ki-67 showed a wider spread in the distribution of percent cases (FIG. 3A), suggesting tremendous diversity in these specimens. In contrast, DCIS specimens with an IHC score of 1 for SOX9 and Ki-67 showed much smaller spread. IDC specimens, on the other hand, showed little variation in percent cases, irrespective of the IHC score across the board (FIG. 3B), suggesting less diversity within this subgroup (FIG. 3A). Whether this diversity predicts evolution to poorly differentiated breast cancer could not be assessed because the samples were not matched pairs of DCIS and IDC.

### Example 3

This Example discusses the results set forth in Example 2.

The results set forth above contain three observations that provide a clear rationale for using SOX9 as a biomarker for identifying poor prognostic invasive breast cancers. The first observation is based on gene expression analysis of publicly available breast cancer databases. This analysis revealed that SOX9 expression is significantly associated with the estrogen receptor negative phenotype, higher tumor grade and poor overall survival. The second observation indicates that SOX9 protein is undetectable using IHC in normal breast tissue but is significantly over-expressed in some invasive ductal carcinomas and lymph node metastasis specimens. Third, and finally, unlike ADH, where SOX9 expression is nuclear, DCIS & invasive ductal carcinomas show cytoplasmic expression of SOX9 that significantly correlates with Ki-67 expression in the IDC specimens. These observations indicate a hitherto unknown but important functional role for SOX9 in a subset of breast cancers and indicate that SOX9 localization can be used as a biomarker to mirror its functional status in human tumors.

Invasive breast cancer evolves through alterations in many regulatory pathways over time. Thus, the key to finding effective measures of intervention would be to identify biomarkers that not only change with the earliest changes in breast epithelium, but also continue to reflect the tumor's transition to invasive phenotype. The results set forth herein show that SOX9 can serve as such a biomarker. For example, its expression is evident in ADH, the earliest lesion of breast cancer. Previous studies have shown that some ADH give rise to low-grade/non-comedo DCIS, while the poorly differentiated DCIS are thought to evolve from occult precursors (Allred et al., Endoc Relat Cancer 8:47-61 (2001)). These observations, together with the result herein that SOX9 was nuclear in the ADH sample, suggest that SOX9 may be associated with those ADH that progress to well differentiated DCIS, and upon accumulation of additional genetic hits, give rise to invasive breast cancer. Humanized models of breast tumor progression (Behbod et al., Breast Cancer Res 11:R66 (2009); Miller et al., J Natl Cancer Inst 92:1185-6 (2000); Miller et al., J Natl Cancer Inst 85:1725-32 (1993)) may help elucidate SOX9's role in the transition of ADH to DCIS, and then to invasive carcinoma.

The studies reported in the previous Example show that SOX9 displays yet another characteristic of a good prognostic biomarker—its ability to reflect the inherent aggressiveness of a tumor. This is best revealed by SOX9's n widely different expression levels, that range from undetectable in normal tissues to nuclear in benign to cytoplasmic overexpression in DCIS, IDC, and metastatic breast cancers. Such variable expression levels of SOX9 in normal and cancer, pre-invasive and invasive carcinomas provide an excellent rationale to use SOX9 as a surrogate to identify potentially aggressive and metastatic breast cancers. However, cytoplasmic expression of SOX9 in almost a similar percentage of DCIS specimens as IDCs may raise the concern that SOX9 expression levels alone may be insufficient to unambiguously predict progression to invasive disease. This may be reconciled, considering that previous studies have shown that DCIS are propagated to IDCs in a manner independent of progression to invasion (Allred et al., Clin Cancer Res 14:370-8 (2008), and that DCIS are obligate precursors for invasive breast cancers (Allred et al., Endoc Relat Cancer 8:47-61 (2001); Gupta et al., Cancer, 80:1740-5 (1997); Hu et al., Cancer Cell 13:394-406 (2008)). All of these studies support the position that changes in expression patterns of SOX9 alone should be sufficient to predict a tumor's propensity to evolve into invasive breast cancer.

This study of random assorted breast tumors, representing various stages of breast cancer, highlights other important facets of SOX9 such as its preferential expression in ductal carcinoma specimens. Similarly, the SOX9 expression pattern changes as the disease evolves. Thus, SOX9's ability to demarcate the earliest changes in the breast epithelium along with its ability to distinguish the pathobiology of tumors originating from distinct types of epithelial cells, such as ductal and lobular epithelial cells, should lead to new approaches for better management of these cancers. Furthermore, the strong association between higher expression of SOX9 and ER− phenotype in this study, and the knowledge that ER status has often been clinically used as a predictive marker for hormonal therapy (Osborne and Schiff, J Clin Oncol, 23:1616-22 (2005)), strongly suggests that combining the analysis of SOX9 and the presence or absence of ER would substantially enhance the prognostic power of these markers, as together they would more accurately reflect the natural history of the disease.

One of the most intriguing results of the present study that highlights the importance of assessing SOX9 expression in human IDCs is the observation that mostly tumors with cytoplasmic accumulation of SOX9 showed Ki-67 expression (FIGS. 3A and B), indicating that this phenotype may represent a non-mutational mechanism for abrogation of SOX9's growth arrest function. While this association is significant, intertumoral diversity was equally apparent and most pronounced in DCIS, consistent with those reported earlier. (Allred et al., Clin Cancer Res 14:37-08 (2008)). These results demonstrate the relevance of the findings herein for human breast tumor progression and raise the possibility that cytoplasmic SOX9 may represent a gain of function SOX9 allele. Therefore, unlike Ki-67, that detects only proliferating cells, serial staining for SOX9 should help identify tumors that would have a higher propensity to pursue an aggressive course.

The finding herein that SOX9 over-expression correlates with reduced overall survival is consistent with those of Lu et al. (Am J Clin Pathol, 130:897-904 (2008)) for colorectal cancer patients and substantiate the hypothesis herein that SOX9 directly contributes to breast cancer progression. However, by comparing only the top and bottom 10% SOX9 expressors and ignoring the remaining 80% of the samples, the analysis might suffer some minor bias. Such inherent biases may be minimized using larger data-sets that compare the groups as tertiles of expression with sufficient number of samples per group, and per tertile. A larger sample size study would also help determine how ER status may be influencing the survival outcome in these patients. This is important because of the finding herein SOX9 is over-expressed in ER− breast cancers that are known to have poor overall survival. Nonetheless, if cytoplasmic expression of SOX9 is an indicator of possible progression to invasive disease, relative assessments of nuclear versus cytoplasmic expression of SOX9 in breast cancer patients prior to and after therapies, and stratification of the data based on hormone receptor status would be helpful in determining whether cytoplasmic up-regulation of SOX9 results in a more malignant phenotype of mammary tumors with reduced overall survival. The fact that SOX9 protein is unstable with a t_{1/2} of 3.6±0.22 h, yet a sizable proportion of DCIS, IDCs and lymph node metastasis samples show a strong cytoplasmic localization of the protein that is reminiscent of cytoplasmic sequestration of p53 in human tumors that also results in worse prognosis. However, unlike p53, there are no data available to suggest that SOX9 locus is amplified or mutated in breast cancer or other cancers. This poses the question whether SOX9 locus/gene undergoes mutation, and whether the loss of its nuclear functions up-regulates the expression of invasion and metastasis genes.

Although the present studies suggest that other SOX family members may not compensate for SOX9 function, conservation of the HMG domain and high homology with the E-group members indicates possible functional overlap with other SOX family of genes. The reverse transcriptase-polymerase chain reaction data from human mammary epithelial cells and breast cancer cell lines confirm previously reported expression of SOX2 and SOX4 in breast cancer cells. However, none of the cell lines under investigation express the other two E group gene (SOX8 or SOX10) mRNAs, suggesting only SOX2 or SOX4 may partner with SOX9, or, share common functional targets. However, both SOX2 and SOX4 are single exon genes, while SOX9 encodes a triple exon gene, and, apart from sharing the conserved HMG domain with these two members, it has additional flanking sequences that may allow it to interact with many additional proteins to form diverse transcriptional complexes. More importantly, in the present study, only SOX9 mRNA is upregulated in a manner that was representative of the observations in human tumors, suggesting that although these genes may be co-expressed, they may have little to no functional overlap during cancer progression, especially in influencing the meta metastatic phenotype of breast cancer cells.

In conclusion, the data reported herein indicate that SOX9 contributes directly to or is at least a marker for the poor clinical outcomes associated with invasive breast cancer. Thus, monitoring SOX9 expression over the course of the disease and modulating its signaling, particularly in cases where it is preferentially localized in the cytoplasm, is a useful way to determine the growth characteristics and prognosis of a subset of metastatic breast cancers.

### Example 4

This Example sets forth the materials and methods used in the studies reported in Examples 5 and 6.

Reagents:

Stock solutions of TSA (common name Trichostatin A; [R-(E,E)]-7-[4-(Dimethylamino)phenyl]-Nhydroxy-4,6-dimethyl-7-oxo-2,4-heptadienamide; Sigma-Aldrich (St. Louis, Mo., catalog no. T-8552), and leptomycin B in 70% (v/v) methanol (Sigma-Aldrich, catalog no. L2913) were stored at −20° C. Antibodies to SOX9 (Chemicone unit of Millipore, Billerica, Mass., catalog no. AB5535), actin (Sigma-Aldrich), p21 and Acetylated Lysine (Cell Signaling, USA) were used.

Collection and Processing of Mouse Mammary Glands from Embryonic and Adult Mice:

Embryonic, pre-pubertal, pubertal, pregnant, and lactating mammary gland tissue from wild type females of CD1 background were collected in Dr. Yiping Chen's laboratory, fixed in 4% paraformaldehyde and processed for embedding and sectioning as described previously (Chakravarty et al., Mol Endocrinol 17:1054-65 (2003))). Staging of embryos was done by taking the morning of vaginal plug detection as embryonic day 0.5 (E0.5). Pubertal glands were collected from 5 wk and 12 wk mature virgin mice. To assess the effect of pregnancy, inguinal glands from pregnancy day 12 mice were harvested. Lactating glands were collected from females nursing their pups for 2 days post partum. Mice were fed a conventional diet ad libitum and maintained at 21-22° C. with a 12-h light, 12-h dark cycle. Animal protocols were approved by the Animal Care and Use Committee of Tulane University and were conducted in accordance with NIH guidelines. All animals were maintained in accordance with the provisions of the Guide for the Care and Use of Laboratory Animals and the Animal Welfare Act.

Immunohistochemistry.

Tissues were fixed with 4% paraformaldehyde, dehydrated through the ethanol series, and paraffin embedded. Five-micrometer sections were baked on an infrared hot plate for a minute, deparaffinized with xylene, and rehydrated with ethanol series. Heat-induced antigen retrieval was performed in 10 mM citrate buffer for 15 min followed by slow cooling to room temperature. Endogenous peroxidase activity was blocked by incubating the sections in 3% hydrogen peroxide solution for 5 min. All incubations were performed at room temperature, and all washings were performed with PBST (Ca⁺⁺Mg⁺⁺ free PBS+0.05% Tween® 20) unless otherwise stated. Endogenous biotin was blocked using the Avidin/Biotin blocking kit according to the manufacturer's instructions (Vector Laboratories, Inc., Burlingame, Calif.). Slides were then incubated with SOX9 antibody (1:2000 dilution in Tris-buffered saline+1% BSA) for 1 h, biotinylated secondary antibody (1:250) for 30 min, and then horseradish peroxidase-labeled avidin (1:200) for 30 min. For negative control, slides were incubated with pre-adsorbed antibody. Detection was achieved by incubation with diaminobenzidine (DAKO Corp., Carpinteria, Calif.) until the brown color was visible under the microscope. Slides were counterstained with CAT hematoxylin for 30 sec, dehydrated, and mounted using Permount (Sigma, St. Louis, Mo.). Human adult skin sections served as the positive control of SOX9 staining. Additional negative control was obtained by incubating the slides with purified rabbit immunoglobulin (The Jackson Laboratory, Bar Harbor, Me.). The anti-SOX9 antibody used in the study recognizes a 68-75 kDa full-length human SOX9 protein in western blots and cross-reacts with SOX9 protein.

Cell Culture.

MCF7 human breast adenocarcinoma cells, MDA-MB-231, ZR-75-1 & MCF10A cells were obtained from the American Type Culture Collection (Manassas, Va., catalog nos. HTB-22, HTB-26, CRL-1500, and CRL-10317, respectively). MCF7 cells were maintained in Eagle's Minimum Essential Medium (MEM) with Earle's balanced salt solution, containing 10% fetal bovine serum, 100 U/ml penicillin, 100 μg/ml streptomycin, 2 mM L-glutamine, 0.1 mM non-essential amino acids, 1 mM sodium pyruvate and 0.01 mg/ml bovine insulin. MDA-MB-231 & ZR-75-1 cells were cultured in RPMI supplemented with 10% (v/v) fetal calf serum, 2 mM L-glutamine, 1× Antibiotic Antimycotic solution. MCF10A cells were cultured in MEGM media purchased from Lonza Inc. (Allendale, N.J.). Growth conditions were kept at 37° C., with 5% CO₂/95% humidified air.

Immunofluorescence Microscopy for Cells Cultured on Coverslips.

Cells were grown on sterile glass coverslips in 6 well plates to 30%-40% confluence in their respective media, serum starved for the next 24 h treated with 1 nM all trans retinoic acid, 500 nM TSA or 5 ng/mL Leptomycin B for 4 h and then with 10% FBS for 2-3 h. Coverslips were washed three times in PBS and fixed in 4% paraformaldehyde for 30 minutes. After washing the coverslips again with PBS three times, they were blocked with 10% whole goat serum (Santa Cruz Biotechnology, Inc., Santa Cruz, Calif.) for thirty minutes. Cells were stained with anti-SOX9 (1:1,000 dil) antibody or anti-DDK tag antibody (OriGene Technologies, Inc., Rockville, Md.) and visualized using alexa 488-conjugated or 594-conjugated secondary (1:500 dilution) antibody (Molecular Probes®, Invitrogen Corp., Carlsbad, Calif.) as described previously (Chakravarty et al., Exp Biol Med (Maywood), 234:372-86 (2009)). For analysis, cells were visualized with a Nikon N300 microscope (Nikon Instruments Inc., Melville, N.Y.). Filter sets for FITC and DAPI were used to capture images that were then analyzed with Elements software. To establish that response to growth arrest signals was dependent on SOX9's cellular localization, both cell lines were also cultured on coverslips, serum starved for 24 h, and subsequently exposed to 10% FBS and stained with SOX9 antibody as described above.

Reporter Genes:

The Col2a1 reporter gene (Lefebvre et al., Mol Cell Biol, 17:2336-46 (1997)) was kindly provided by Prof. Benoit deCrombrugghe's laboratory. The beta catenin reporter set containing the plasmids TOP-flash and FOP-flash were made available by Prof. Randall Moon's laboratory.

Transfection Experiments:

For transfection, MDA-MB-231 cells were cultivated at 2.5×10⁵ cells per well in 6-well plates. Twenty four hours later, transfection was performed using lipofectamine 2000 (Invitrogen) according the manufacturer's instruction. Cells in 6-well plates were transfected with 0.250 μg DNA of Col2a1 reporter or 0.300 μs DNA of Top-flash or Fop-flash reporters, 10 ng of Renilla luciferase and 1.0 μg of wild type DDK tagged SOX9 per well. Top-flash or Fop-flash reporter transfected wells were treated with 15-ng/ml recombinant wnt3a protein (R&D Systems, Inc., Minneapolis, Minn.) after 24 h of transfection. All transfected cells were lysed directly in Promega's Dual Luciferase lysis buffer and collected with a cell scrape. Luciferase measurement was performed using a Optocomp I luminometer (MGM Instruments, Inc., Hamden, Conn.). Cellular localization of wild type DDK tagged SOX9 was detected using immunofluorescence as described above.

Gene Expression Profiling Using RT² Profiler™ PCR Array.

MDA-MB-231 cells with or without TSA treatment were harvested for total RNA using Trizol (Invitrogen) and reverse transcribed using RT² first strand kit from SABiosciences (Frederick, Md.). Real-Time PCR was performed according to the RT² Profiler PCR Array User manual (SABiosciences) using SYBR Green PCR Master Mix for the CFX96™ Real-Time PCR detection system from Bio-Rad (Bio-Rad Laboratories, Inc., Hercules, Calif.). The PAHS-090C PCR Array was repeated twice for each cDNA sample and the data were analyzed using Excel-based PCR Array Data Analysis Templates from SABiosciences.

The average Ct value of each gene obtained from duplicate experiments was used to calculate its expression value, which was expressed as 2−ΔCt (ΔCt=CtGOI−Ave CtHKG; GOI=gene of interest, HKG=housekeeping gene, Ave CtHKG=average Ct value of five housekeeping genes).

MTT cytotoxicity assay: MDA-MB-231 or MCF7 cells were plated onto 96 well plates at a density of 2.5×10³ cells/well. Cells were allowed to attach overnight and serum starved for the next 24 h. Following starvation, cells were treated with increasing concentration of Trichostatin A (50-500 nM) or Leptomycin B (0.5-10 ng/ml) and allowed to grow for next 24 h. At the end of 24 h drug treatment, media was supplemented with an equal volume of media with 20% FBS. An MTT assay was used to determine cell viability after exposure to test compounds for 48 h, including the 24 h of incubation with 10% FBS. Briefly, 10 μl of MTT reagent (5 mg/ml in PBS) was added to each well, the plate was incubated for 2-4 h to allow for the formation of formazan crystals, the media was aspirated from all wells and crystals were dissolved with 150 μl DMSO. The optical densities were measured at 540 nm on a BioTek uQuant microplate spectrophotometer (BioTek Instruments, Inc., Winooski, Vt.) and the percent of surviving cells was calculated with respect to control or untreated cells. For the modified MTT assay, the classical MTT reagent was replaced with WST-8, which produces a water soluble formazan dye upon bioreduction. For this assay, at the end of drug treatment, cells were incubated with 10 μl of WST-8 per 100 μl media/per well for 2-3 h. O.D. at 450 nm was measured to determine cell viability in each well.

Cell Cycle Analysis:

Cells were grown in 6 well plates to 30%-40% confluence in their respective media, serum starved for the next 24 h and treated with an equal volume of 20% FBS media and allowed to grow for next 24 h as described earlier. Cells were harvested from 6 well dishes and pipetted into a homogenous suspension. In brief, the cell suspension (about 10⁶ cells/ml) was centrifuged for 5 min (116 μg) and 3 ml of 70% ethanol at 4° C. was then added slowly while the container was shaken. After overnight fixation, cells were washed in PBS and stained with a mixture of 30 μg/ml PI (Propidium Iodide; Sigma), and 10.0 μg/ml RNase A at 37° C. for 30 min. The DNA cell cycle analysis was performed on a Beckman-Coulter Epics FC500 flow cytometer running CXP software (Beckman Coulter, Inc., Miami, Fla.). Fluorescence data were obtained by accumulating 20,000 events per sample and then cell cycle modeling was performed using ModFit LT v3.2 (Verity Software House, Topsham, Mass.).

Immunoblot and Immunoprecipitation Analysis:

Asynchronous cell populations at a density of 50-60% in 6-well plates were serum deprived for 24 h, treated with TSA (500 nM), or Leptomycin B (5 ng/ml) for the next 4 h and then cultured in the presence of 10% FBS for 2-3 h before harvesting. Immunoblotting was performed by solubilizing cells in RIPA buffer, electrophoresing on 10% SDS-PAGE gels, incubating with primary antibodies for 1-2 h at room temperature, and detecting primary antibodies using HRP-conjugated secondary antibodies (1:30,000 dilution, Santa Cruz Biotechnology Inc.) and Amersham® ECU® (GE Healthcare Bio-Sciences Corp., Piscataway, N.J.) as described in Wang et al., Clin Cancer Res., 12:4755-65 (2006). Western blots were quantified by calculating an integrated density value (IDV) for each band using Bio-Rad's Gel Doc™ system and normalizing to the IDV of actin. For immunoprecipitation, total protein lysates were centrifuged at 14,000×g for 15 minutes at 4° C. Supernatants were then transferred to fresh centrifuge tubes and precleared with 50 μl of protein A Sepharose™ bead slurry (50%) per 1 ml of cell lysate. Incubation was continued at 4° C. for 30 minutes on a rocker or orbital shaker. Protein A beads were then collected by centrifugation at 14,000×g at 4° C. for 5 minutes. Protein concentration of the saved supernatant was determined after removing the beads. Approximately 500 μg to 1 mg of protein from each sample was transferred to fresh centrifuge tubes and diluted to 1 ml with ice cold PBS to reduce the concentration of the detergents in the buffer. The diluted lysates were incubated with 2 μg/mg protein anti-SOX9 antibody and rotated overnight at 4° C. Immuno complexes were captured with slurry of protein A-Sepharose™ CL-4B (Sigma) pre-washed with BSA through rotations for 1 h at 4° C. and washed three times with ice-cold modified RIPA buffer (Tris-HCl: 50 mM, pH 7.4, NP-40:1%, Na-deoxycholate: 0.25%, NaCl: 150 mM, EDTA: 1 mM, PMSF: 1 mM, NaF: 1 mM). Washed Sepharose™ beads were resuspended in 60 μl RIPA buffer with SDS dye and resolved on 10% SDS-PAGE gels. Blotted proteins were immunoprobed with antilysine antibody to detect the levels of acetylated SOX9.

Statistical Analysis:

Statistical significance was examined using the Student t-test or Fisher's exact test. Values of p<0.05 were considered significant. Values were expressed as means+/−SEM.

### Example 5

This Example sets forth the results of further studies underlying the present invention.

Both embryonic and adult mouse mammary glands stained strongly for SOX9 in the nucleus of ductal cells. However, some of the stromal cells of the E14.5 embryonic buds also stained strongly for SOX9 in the nucleus. The stromal staining was less evident in E17.5 mammary bud. Interestingly, intense nuclear staining was restricted to cells lining the ducts while the remaining cells displayed only background levels of staining Ductal staining was also evident in pubertal and pregnant glands. The fully differentiated lactating mammary glands on the other hand lacked any detectable staining in the nucleus or the cytoplasm. Epithelial cells from normal human adult breast tissue also failed to display any detectable staining either in the nucleus or the cytoplasm. Accordingly, in vitro cell culture models were considered that could be used to understand the significance of SOX9 localization in the cytoplasm and its role in human breast cancer.

Decreased Nuclear and Increased Cytoplasmic Localization of SOX9 in Some Breast Cancer Cell Lines:

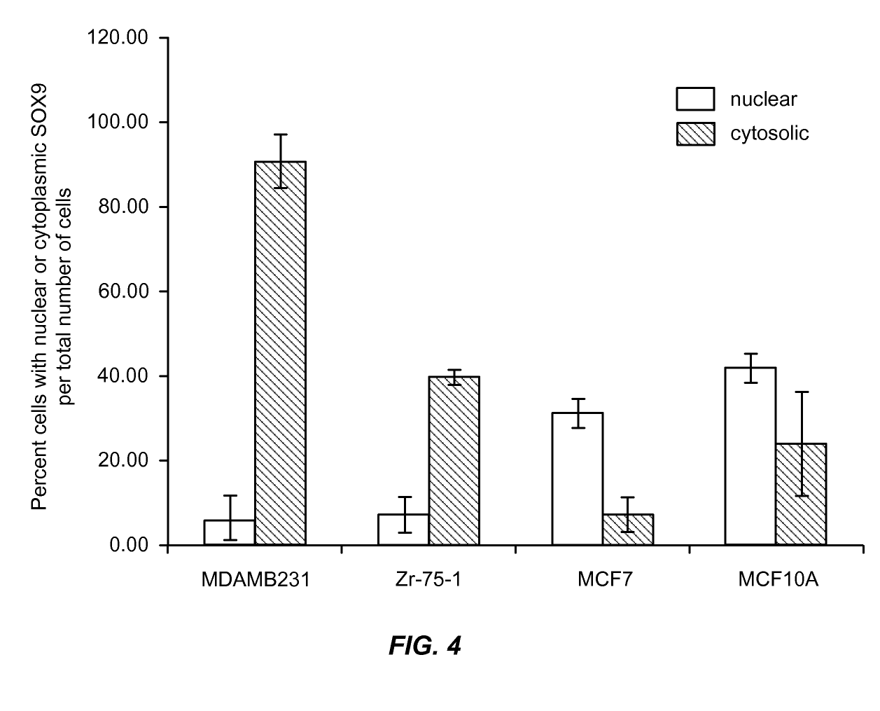

Consistent with the studies reported in Examples 1-3 demonstrating cytoplasmic localization of SOX9 in human breast tumors, some breast cancer cell lines also displayed cytoplasmic localization of SOX9. In particular, MDA-MB-231 and ZR-75-1 cells had more pronounced cytoplasmic compartmentalization of SOX9 as compared to MCF-7 cells or cells derived from a spontaneous human breast tumor (MCF10A). The mean number of cells with cytoplasmic SOX9 in MCF10A cells and hormone sensitive breast cancer cell line MCF-7 per three random fields were 24% and 8% respectively, whereas for the highly metastatic breast cancer cell line MDA-MB-231 it increased to 91%, an escalation of 67-83%, respectively (FIG. 4). In contrast, the corresponding number of cells with nuclear SOX9 for the first two cell lines were significantly higher (p=0.0007; Fisher's exact test, Table 2) for these cell lines.

Additionally, as some transcription factors undergo cleavage before being translocated to the nucleus, total extracts from these breast cancer cell lines were also analyzed by western blotting to see if SOX9 protein underwent any cleavage to facilitate its nuclear translocation in MCF7 or MCF10A cells. None of the cell lines, however, showed any evidence of truncated SOX9 protein irrespective of their hormonal and metastasis potential.

TABLE 2
Cellular localization of SOX9 in select breast cancer
cell lines by immunohistochemistry

| Sample | | | | |
|---|---|---|---|---|
| Localization | MDA-MB-231 | Zr-75-1 | MCF-7 | MCF-10A |
| Avg. Nuclear | 7 | 8 | 35 | 42 |
| SOX9 (%) | | | | |
| Avg. | 91 | 40 | 8 | 24 |
| Cytoplasmic | | | | |
| SOX9 (%) | | | | |

For Table 2: SOX9 staining was scored as follows. Three random fields from three different coverslips for each cell line were photographed at 200× magnification and scored for number of cells with nuclear or cytoplasmic staining. This number was further divided by the total number of DAPI stained nuclei to obtain the percentage of cells with nuclear or cytoplasmic SOX9 staining Cells covering more than 50% of the DAPI stained nuclei with green fluorescent stain were counted as nuclear, whereas cells with cytoplasmic or perinuclear staining were counted as cytoplasmic.

Cells with Cytoplasmic SOX9 Demonstrate Impaired Transcriptional Activation of Two Well Characterized SOX9 Target Reporters.

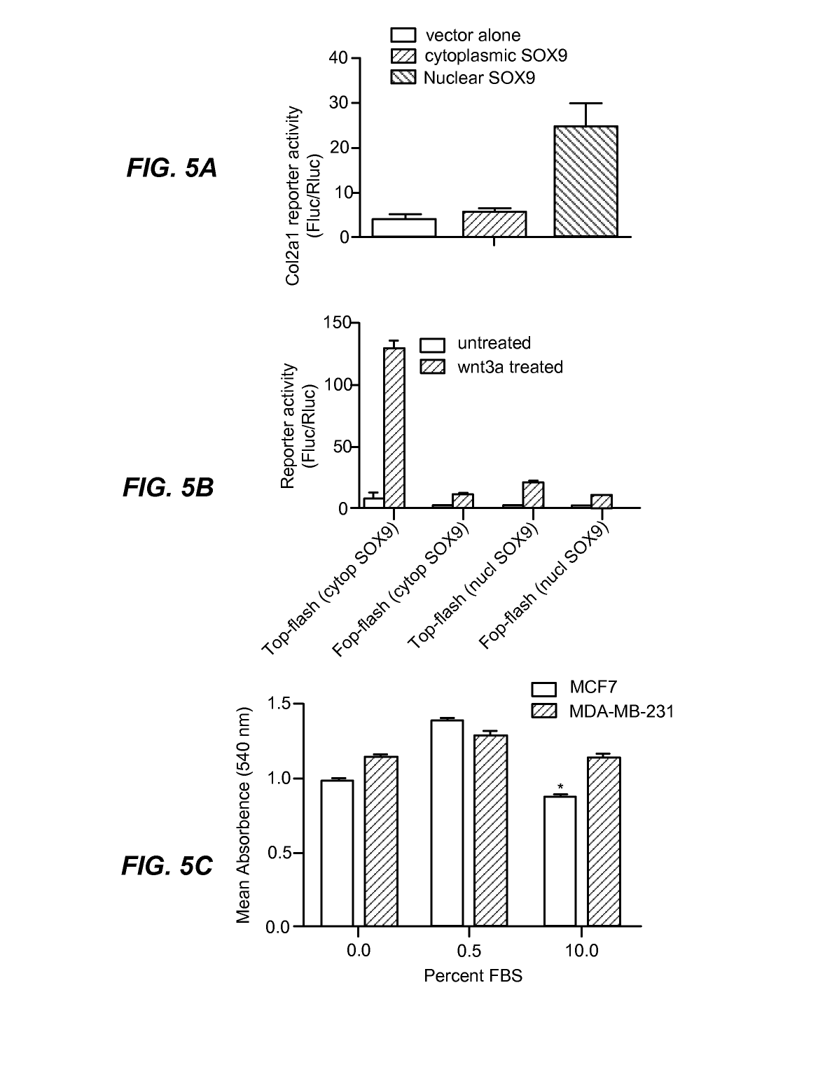

The next investigation was to determine whether cytoplasmic localization of SOX9 results in complete amelioration of its nuclear functions. This was achieved by assaying transcriptional activation of known SOX9 target genes. Since little is known about direct transcriptional targets of SOX9 in breast cancer cells, two well-characterized SOX9 reporters (Col2a1 and beta-catenin) known to be activated or repressed by SOX9 in other cell types were chosen. Both of these genes are expressed in mammary cells. Accordingly, the question posed was whether MDA-MB-231 cells with predominantly cytoplasmic SOX9, completely lose their ability to regulate the activity of Col2a1 and wnt beta-catenin reporters. As shown in FIG. 5A, MDA-MB-231 cells transfected with the Col2a1 reporter show only minimal upregulation (1.4-fold) of the reporter as compared to cells transfected with vector alone. However, co-transfection of the same cells with wild type SOX9 resulted in more than 5-fold induction of the Col2a1 reporter activity (black bars) as compared to the MDA-MB-231 cells with endogenous SOX9 expression (grey bar). Interestingly, unlike the endogenous SOX9 protein, which was cytoplasmic, exogenously transfected SOX9 was localized in the nucleus of these cells. Similar results were obtained with the TOP-FLASH reporter system as well (FIG. 5B). Specifically, although SOX9 is known to inhibit beta-catenin activity, the TOP-FLASH reporter showed 14-fold induction when wnt signaling was activated with recombinant wnt3a protein in MDA-MB-231 cells. Once again, introduction of wild type SOX9 through transfection led to 6-fold lower induction of the TOP-FLASH reporter (compare cytoplasmic SOX9 bars to nuclear SOX9 bars), suggesting cytoplasmic SOX9 protein in MDA-MB-231 cells is incapable of translocating to the nucleus and regulating its target genes. To ensure that this response was wnt driven, these cells were also transfected with FOP-FLASH vector that has the same backbone as the ‘TOP-FLASH’ vector, but the LEF-1/TCF-binding sites of this vector have been mutated. The data indicate that the FOP-FLASH reporter had some basal activity in these cells (FIG. 5B) but unlike the TOP-FLASH reporter, its activity was unchanged between untreated and wnt3a treated cells and was also independent of cellular localization of SOX.

Cytoplasmic Localization of SOX9 Correlates with Abrogation of Cell Cycle Arrest Response of Breast Cancer Cells:

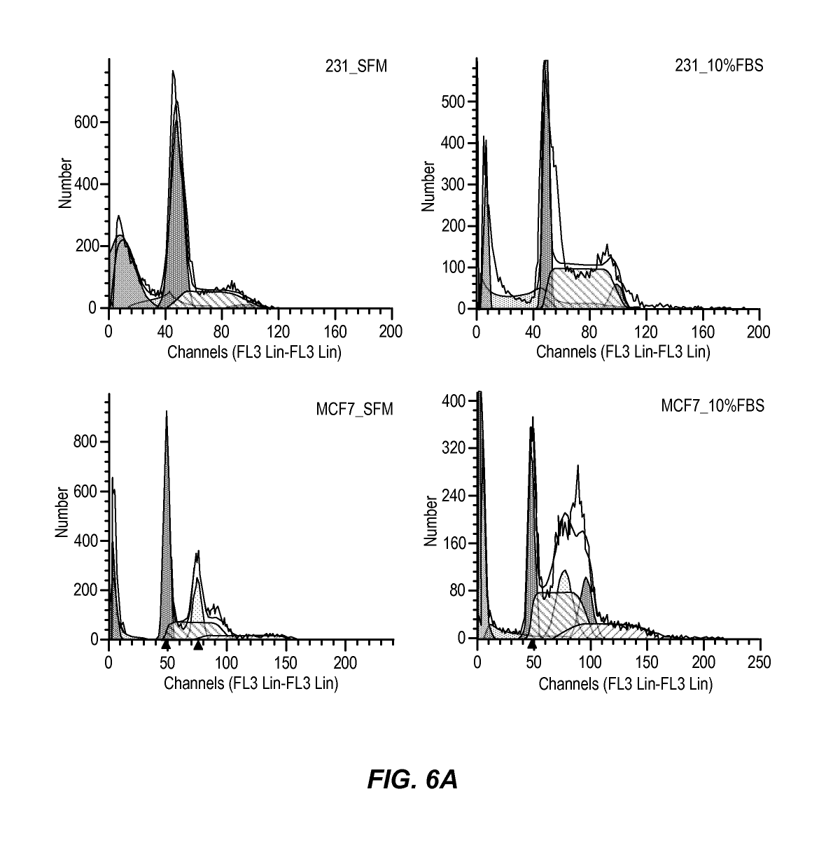

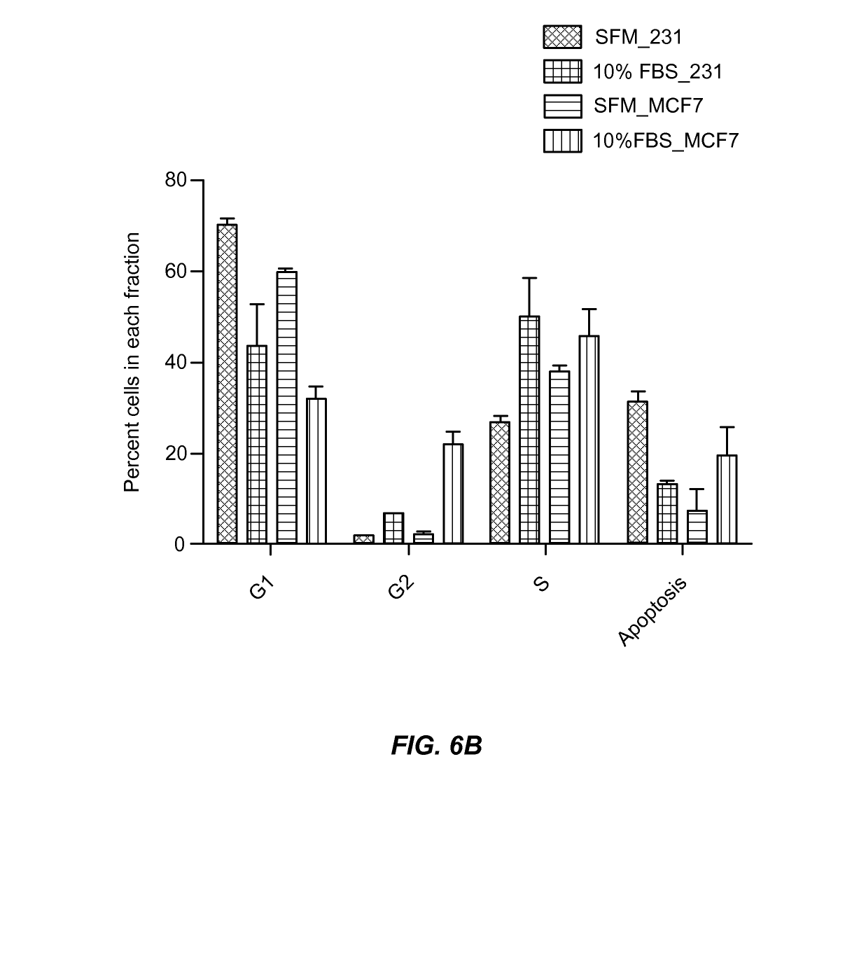

Since SOX9's inability to translocate to the nucleus in response to growth arrest signals may allow cancer cells to continue to proliferate indefinitely, the effect of SOX9 localization on cycle arrest of human breast cancer cells was examined. To test this hypothesis, serum deprived MCF7 and MDA-MB-231 cells were assayed for percent proliferation using the classical MTT assay as described in the Examples. As expected, MDA-MB-231 cells that show cytoplasmic localization of SOX9 continued to proliferate after these cells were released from cell cycle arrest with the addition of serum but the growth rate of MCF-7 cells was significantly inhibited (p=0.026, FIG. 5C) and correlated with increased nuclear localization of SOX9. To further confirm that this was due to changes in cell cycle, numbers of cells in G₁, G₂ and S phase of the cell cycle were determined using flowcytometry. MDA-MB-231 cells that were serum deprived and then exposed to serum had more cells in S phase as compared to MCF-7 cells (FIG. 6A). Specifically, there was a 54% increase in the S phase fraction of MDA-MB-231 cells when these cells were grown in SFM vs. in 10% FBS media. In contrast, MCF-7 cells showed only a 17% increase in the S phase fraction under these conditions (FIG. 6B). The observation that MDA-MB-231 cells failed to show growth inhibition was also substantiated by a corresponding decrease (56%) in the number of apoptotic cells in MDA-MB-231 cells as opposed to MCF-7 cells that registered an increase (45%) instead. Additionally, a much larger fraction of the MCF-7 cells (22%) were arrested in the G₂M phase of the cell cycle as opposed to just 7% in the MDA-MB-231 (FIG. 6B) cell line. It is important to consider though that there are numerous other differences between these cell lines that might account for or contribute to the differences observed in the cell cycle of these two cell lines.

It is also noted that the MCF-7 cell line has 25-30% aneuploid cells and although this aneuploid population showed a trend that was similar to the diploid population of this cell line, data used to calculate the increase in S phase and apoptosis did not take into account the data from the aneuploid fraction of MCF7 cells.

TSA Treatment Rescues the Growth Arrest Response Through Nuclear Accumulation of SOX9 and a Concomitant Increase in p21 Expression and Cell Death:

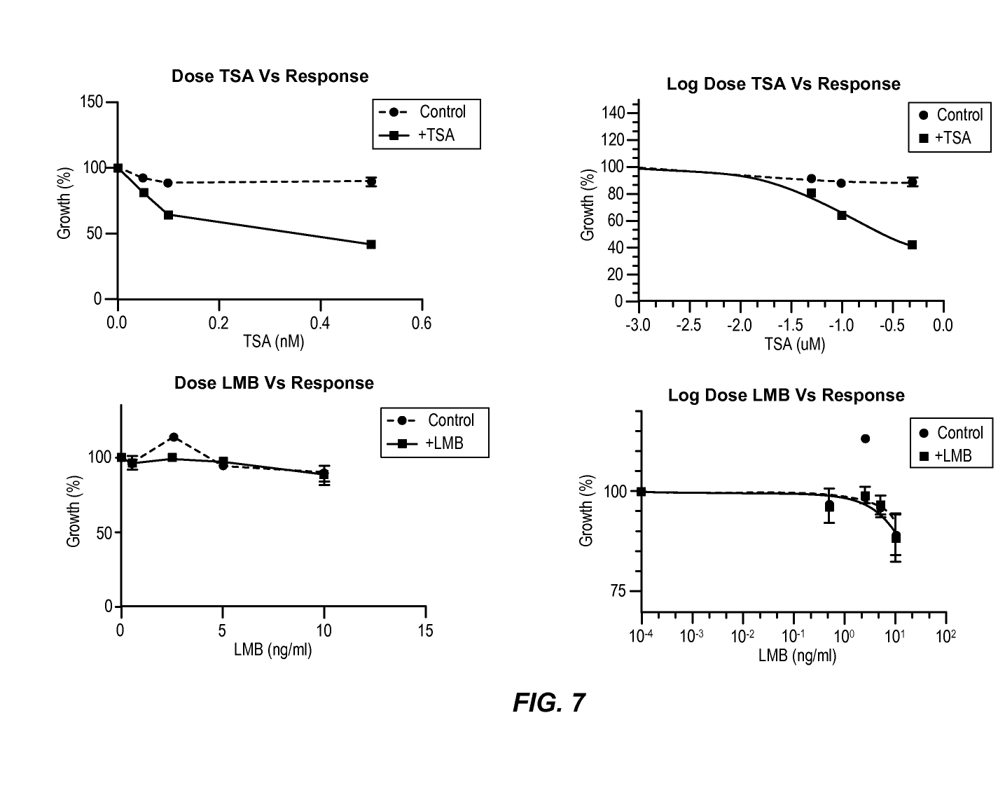

Epigenetic events like DNA methylation and histone acetylation are known to play an important role in nuclear cytoplasmic shuttling of proteins. (McKinsey, et al., Nature, 408:106-11 (2000)). To investigate if SOX9's inability to translocate to the nucleus to induce growth arrest response in MDA-MB-231 cells is due to dysregulated HDAC activity, serum-deprived MDA-MB-231 cells were treated with HDAC inhibitor TSA to see if it would induce nuclear translocation of SOX9. Serum-deprived, TSA treated MDA-MB-231 cells showed a marked increase in nuclear SOX9 staining in response to serum exposure. Interestingly, longer exposure to serum led to complete depletion of nuclear SOX9. However, in the absence of TSA treatment, these cells continued to show only cytoplasmic or perinuclear SOX9 staining. Furthermore, TSA treated cells demonstrated a dose dependent increase in cell death. To consider the alternate possibility that increased nuclear export of SOX9 may also result in loss of growth arrest, serum starved MDA-MB-231 cells were treated with a nuclear export inhibitor leptomycin B (LMB). As shown in FIG. 7, unlike TSA, that showed a dose dependent increase in cell death, LMB was unable to retain SOX9 in the nucleus or induce cell death in MDA-MB-231 cells (FIG. 7, lower left field).

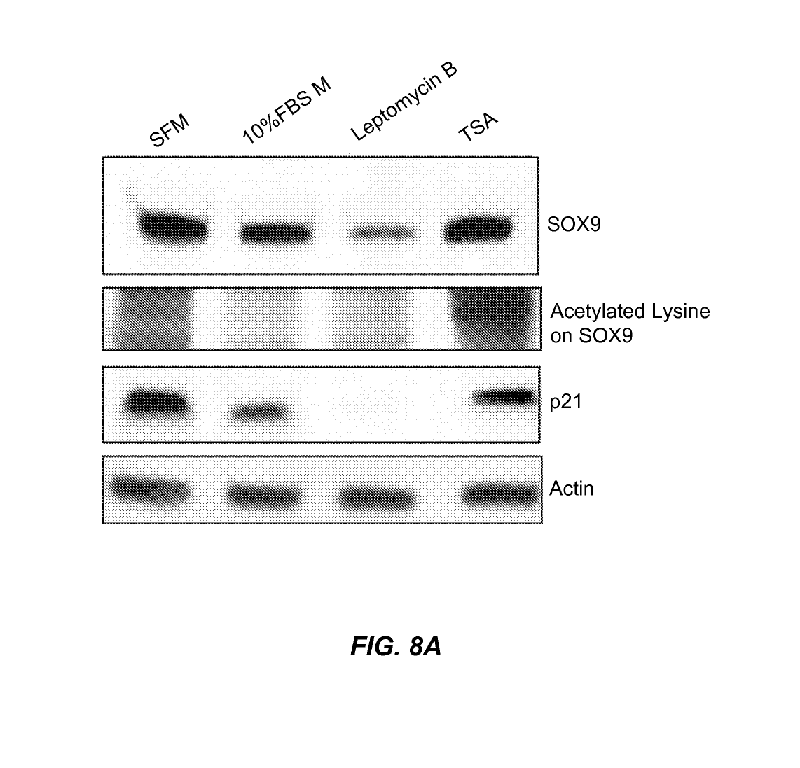

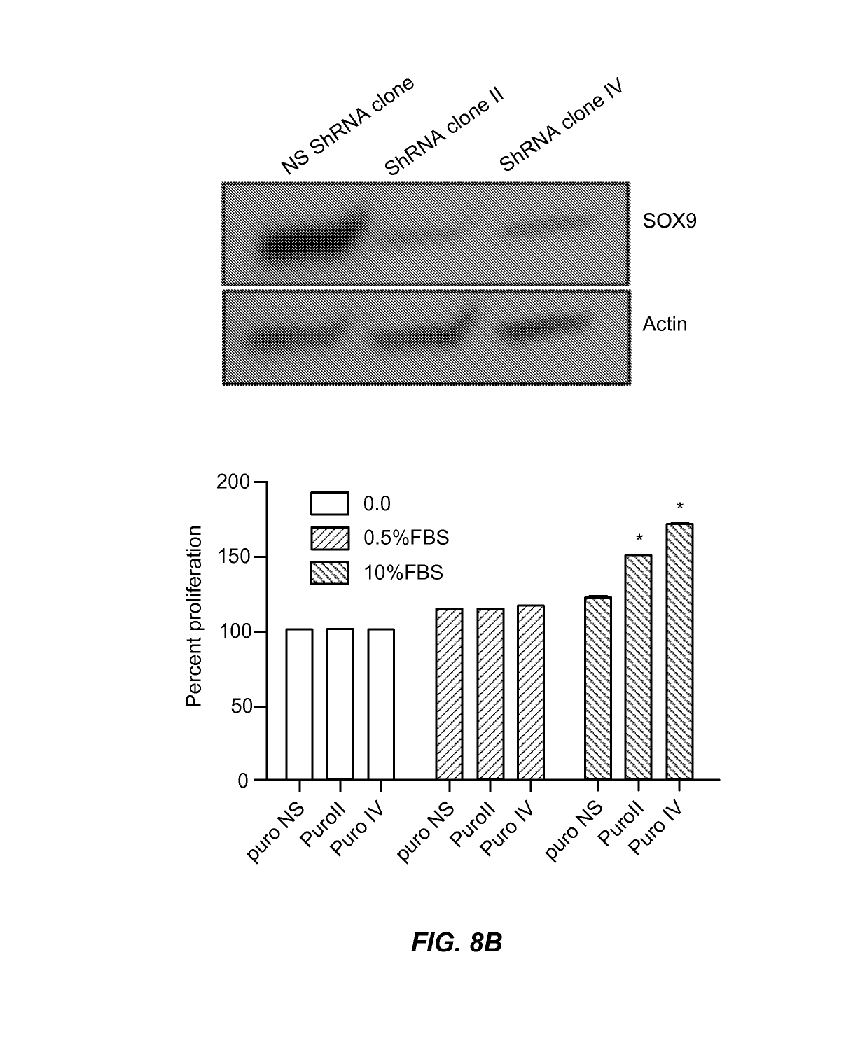

To investigate whether cell death could be attributed to a concomitant increase in p21 expression, total cell lysates from serum starved, TSA treated and unstarved cells were screened for endogenous levels of p21, SOX9 and acetylated SOX9 protein. As shown in FIG. 8A, TSA treatment of MDA-MB-231 cells resulted in a concomitant increase in p21 expression (1.3 fold increase in p21 arbitrary units normalized to actin level) when compared to cells grown in 10% FBS medium. Once again, LMB treatment was not accompanied by higher p21 expression (0.08 fold change as compared to cells grown in 10% FBS media). A similar trend was observed for endogenous SOX9 protein expression (1.2 fold increase in SOX9 arbitrary units normalized to actin level) in response to TSA treatment. Interestingly, serum starvation also resulted in higher endogenous levels of p21, SOX9 and acetylated SOX9.

To further investigate whether this remarkable difference in target gene expression in response to TSA treatment and nuclear localization of SOX9 is restricted to a limited number of genes or has genome-wide implications, expression profiles of MDA-MB-231 cells with or without TSA treatment were compared. A comparison of the gene expression profile of MDA-MB-231 cells where SOX9 is mainly in the cytoplasm (serum starved and then treated with FBS) to the profile of cells where SOX9 is mostly in the nucleus (serum starved, treated with TSA for 4 h and then treated with FBS) indicated that nuclear localization of SOX9 is accompanied by several fold higher expression of genes involved in growth, development and differentiation and downregulation of migration, motility and cytoskeletal genes, substantiating the in vivo findings in which nuclear expression of SOX9 was found to parallel development and differentiation.

Knockdown of SOX9 in Cells with an Abrogated Growth Arrest Response Further Potentiates their Proliferative Potential:

To investigate whether knockdown of SOX9 messenger RNA restores the growth arrest response of breast cancer cells, SOX9 expression was knocked down using lentiviral short hairpin RNA constructs (shRNA) in MDA-MB-231 cells that had elevated levels of both SOX9 protein and mRNA as compared to the normal human mammary epithelial cells. Two SOX9 shRNA clones and a clone generated with a non silencing shRNA were compared for their proliferative potential using a modified MTT assay. The clones were first tested to ensure knockdown of SOX9 mRNA by monitoring SOX9 protein levels (FIG. 8Ba) in these cells. However, as MDA-MB-231 cells are not growth arrested, the experiment was designed to assess the difference in proliferation rates when all of the above clones were cultured in serum free media versus 0.5% FBS or 10% FBS media. Contrary to the hypothesis, both SOX9 shRNA clones II (150±0.5%) and IV (172±1.0%) had significantly higher proliferation rate (two tailed t-test p<0.0001) as compared to the non silencing shRNA clone (121±1.0%) when grown in 10% serum after 24 h of serum deprivation. It is intriguing to note that knockdown of SOX9 did not affect the proliferation rate of the shRNA clones when the cells were grown in serum free or low serum conditions (FIG. 8Bb).

### Example 6

This Example discusses the results set forth in Example 5.

De-differentiation and loss of cell cycle control are hallmarks of cancer progression and are most often controlled by tissue-specific transcription factors. Therefore, investigations into the function and expression of transcription factors may allow a better understanding of the molecular mechanisms underlying neoplastic transformation. Additionally, to ensure proper cellular function, the spatial distribution of different proteins needs to be delicately regulated and coordinated. But very often this regulation is compromised in cancer cells, resulting in altered cell proliferation and response to apoptotic signals.

The studies reported in Examples 1-3 showed that SOX9 expression was significantly associated with poor prognosis ER negative breast cancer and their metastasis. And, although SOX9 expression was nuclear in hyperplasias, it was localized in the cytoplasm of approximately one third of human invasive ductal carcinomas. The studies reported in Examples 1-3 therefore indicate that depletion of SOX9 in the nucleus of ductal mammary epithelial cells represents a critical step in the neoplastic progression of mammary cells, and may be the favored response of cancer cells to surmount SOX9's growth arrest function.

Further investigation of SOX9 expression during mammary development and in human breast cancer cell lines was also warranted by the possibility that SOX9 plays a dual role in mammary cells. During development, nuclear SOX9 may be required for progression to the terminal differentiated state by inducing growth arrest, whereas its cytoplasmic compartmentalization may enhance its proliferative potential in neoplastic cells. To investigate whether this mechanism is responsible for SOX9 driven cancer progression, studies were conducted to determine if SOX9 expression was indeed nuclear during development, and whether its localization was altered mostly in transformed cells. The observation of localization of SOX9 in the nucleus of mammary cells during early stages of mammary differentiation supports the hypothesis that normal differentiating cells are subject to regulatory cues in a manner that SOX9 localization appears constitutively nuclear during early differentiation except that its expression is down regulated in terminally differentiating lactating cells, when it is undetectable by immunohistochemistry. Whether these observations can be extrapolated to generalize changes taking place during human mammary development remains to be determined.

The next step was to look for in vitro cell culture models that reproduce the observation of cytoplasmic compartmentalization of SOX9 in human breast tumors. Observations in three (MDA-MB-231, ZR-75-1 and T-47D) of the five breast cancer cell lines studied support the position that the regulatory processes linking SOX9 localization, cell proliferation and differentiation are impaired in some breast cancer cells. The observation herein that MCF-7 cells continue to exhibit nuclear SOX9 expression and undergo growth arrest, whereas MDA-MB-231 cells with cytoplasmic SOX9 do not do so, are consistent with this hypothesis. However, the correlation between nuclear SOX9 and more differentiated status of MCF7 cells (as compared to MDA-MB-231 or ZR-75-1) implies that tumors with cytoplasmic SOX9 may be derived from early progenitors. Accordingly, SOX9 staining may provide a reliable index of mammary tumor differentiation status. Nonetheless, since the MCF7 cell line is derived from transformed mammary cells but continues to retain the capacity to growth arrest suggests that transformation alone may not be responsible for cytoplasmic compartmentalization of SOX9 in human breast tumors and cell lines. Additional factors, like cytosolic proteins, other co-regulatory proteins, or hormonal factors may act in concert to regulate SOX9's nucleo cytoplasmic shuttling during malignant transformation. These observations also raise the possibility that SOX9 may have bonafide cytosolic functions that remain suppressed during normal development, but surface in cancer cells, especially in those cells that are arrested in early stages of differentiation.

Two alternative hypotheses emerge for possible cytosolic functions of SOX9. Based on the observations herein in MDA-MB-231 and ZR-75-1 cells, SOX9 may be phosphorylated by AKT in the cytoplasm to induce a proliferative response as has been shown for PTEN-driven cyclin D1 localization in the nucleus (Radu et al., Mol Cell Biol, 23:6139-49 (2003)). The second possibility is that it cross talks with Ras, EGFR or HER2neu receptor signaling to elicit a robust growth response, as has been shown for p21Cip1/WAF1 (Zhou et al., Nat Cell Biol, 3:245-52 (2001)). Alternatively, SOX9 compartmentalization may represent a mechanism to regulate its nuclear functions (like regulating the function of other transcription factors) as has been shown for NFκB, which remains tethered in the cytoplasm by association with its partner IκB, thus masking its nuclear localization signal. Its nuclear entry is then determined by several cellular stimuli, which activate IκB degradation (Baldwin, Annu Rev Immunol., 14:649-83 (1996)). This model suggests that SOX9 may remain tethered to the cytoplasm with partner proteins to block the induction of cell cycle arrest genes, but in the presence of the pro cell cycle arrest stimuli, it translocates to the nucleus once the tethering proteins have been degraded. The observations herein of nuclear SOX9 localization during mammary development in this study lend support to this possibility. The studies herein also imply that regulation of SOX9 protein either by cellular compartmentalization or regulation of its protein levels through nuclear degradation may directly influence morphological differentiation and cell cycle changes in breast cancer cells.

Interestingly, SOX9 may translocate to the nucleus following TSA treatment in cells that fail to growth arrest (e.g. MDA-MB-231). In the present studies, such treatment not only resulted in enhanced nuclear localization of SOX9 within 4 h, but also enhanced acetylation of endogenous SOX9 protein and p21 expression in MDA-MB-231 cells (FIG. 8A). This coincided with a decrease in proliferative potential of these cells as measured by MTT (FIG. 7) and increased expression of development, differentiation, and morphogenesis genes. The upregulation of acetylation of endogenous SOX9 protein with TSA (FIG. 8A) suggests that SOX9 activity may be repressed by histone deacetylation during cancer progression. Additionally, since SOX9 nuclear localization or cell death were unaffected by LMB treatment, the loss of SOX9 function in cancer cells may be via nuclear import rather than nuclear export mechanisms. Nonetheless, the observation of nuclear translocation of SOX9 with TSA treatment in MDA-MB-231 cells point to an interesting mechanism for inducing growth arrest in ER-negative breast cancer cells, a mechanism that goes far beyond its known role of inducing cell cycle arrest in estrogen receptor-positive breast cancer cells.

Overall, this study showed that SOX9 localization is differentially regulated in normal and breast cancer cells, thus suggesting that morphological cues or cell cycle changes may facilitate SOX9 nuclear expression during mammary morphogenesis, and that loss of this regulation through cytoplasmic compartmentalization may promote breast cancer growth. Furthermore, cytoplasmic localization of SOX9 in MDA-MB-231 breast cancer cells was associated with their inability to growth arrest in response to serum deprivation or retinoic acid treatment. This could be rescued by TSA treatment, which was able to induce nuclear translocation of SOX9 and enhanced cell death, suggesting that transcriptional repression through direct or indirect interaction with HDACs plays a role in SOX9 mediated growth arrest. These findings are supported by enhanced acetylation of endogenous SOX9 protein and p21 expression in response to TSA treatment.

In conclusion, the data from the present study supports a causative role for SOX9 in breast cancer through loss of its growth arrest function, or through gain of cytoplasmic functions that may initiate hitherto unidentified signaling pathways that promote breast cancer cell proliferation. Using mouse mammary glands and different breast cancer cell lines, the studies herein show that cells with nuclear or cytoplasmic SOX9 respond differently to differentiation or cell cycle specific cues. These observations concur with the studies reported in Examples 1-3 demonstrating that cytoplasmic expression of SOX9 is significantly associated with poor prognosis breast cancers.

### Example 7

Samples of head and neck cancers and of normal oral epithelium and of thyroid cells were examined for the presence of cytoplasmic SOX9. Head and neck cancers that were highly metastatic were found to have much higher expression of cytoplasmic SOX9 than samples from normal tissues. As shown in Table 3, cytoplasmic SOX9 levels were generally several fold higher in highly metastatic oral squamous cell carcinomas and metastatic anaplastic and follicular thyroid carcinoma cell lines than in normal oral epithelium or normal thyroid cells.

TABLE 3
Levels of SOX9 in metastatic head and neck cancer cell lines
compared to normal cell lines.

| | Fold change | | |
|---|---|---|---|
| | over normal | Squamous cell | Fold change over |
| Thyroid cancer cell | (Arbitrary | carcinoma | normal |
| lines | Units) | cell lines | (Arbitrary Units) |
| Normal | 1 | NOE | 1 |
| Thyroid | | (normal | |
| | | tissue) | |
| ARO | 3.072266 | DM14 | 5.877444 |
| WRO | out of range | TU167 | 3.082758 |
| ATCA | 6.92822 | JMAR | 3.39762 |
| KAT4 | 3.520458 | OSC19 | 5.828355 |
| C643 | 4.483384 | | |
| TPC1 | undetectable | | |
| NPA187 | 3.158326 | | |
| FTC236 | 1.527365 | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of screening cancer cells in a sample for aggressiveness, said method comprising detecting the presence or absence of cytoplasmic SOX9 in said cancer cells, wherein the presence of cytoplasmic SOX9 is an indication the cancer is more aggressive and the absence of cytoplasmic SOX9 is an indication the cancer is less aggressive, provided said cancer cells are not from a solid pseudopapillary tumor or melanoma.

2. The method of claim 1, wherein said sample is from a human patient.

3. The method of claim 1, wherein said detection of cytoplasmic SOX9 is by immunohistochemistry.

4. The method of claim 1, wherein said detection of cytoplasmic SOX9 is by immunofluorescence.

5. The method of claim 1, wherein said detection of cytoplasmic SOX9 is by western blotting.

6. The method of claim 1, wherein said detection of cytoplasmic SOX9 is by enzyme-linked immunosorbent assay.

7. The method of claim 1, wherein said cancer cells are breast cancer cells.

8. The method of claim 7, wherein said breast cancer cells are ductal carcinoma cells.

9. The method of claim 7, wherein said breast cancer cells are not invasive lobular carcinoma cells.

10. The method of claim 1, wherein said cancer cells are head and neck cancer cells.

11. The method of claim 10, wherein said head and neck cancer cells are squamous cell carcinoma cells.

12. A method of screening for aggressiveness cancer cells having cytoplasm and a nucleus, said method comprising testing both said cytoplasm and said nucleus of said cells for the presence of SOX9, wherein the presence of SOX9 in said cytoplasm of said cells but not in said nucleus of said cells, or the presence of SOX9 in said cytoplasm of said cells in a quantity greater than SOX9 is present in said nucleus of said cells, is indicative of greater aggressiveness and the absence of SOX9 in the cytoplasm of said cells is indicative of lower aggressiveness,
provided said cancer cells are not from a solid pseudopapillary tumor or melanoma.

13. The method of claim 12, wherein said cancer cells are from a human patient sample.

14. The method of claim 12, wherein said cancer cells are breast cancer cells.

15. The method of claim 14, wherein said breast cancer cells are ductal carcinoma cells.

16. The method of claim 14, wherein said breast cancer cells are not invasive lobular carcinoma cells.

17. The method of claim 12, wherein said cancer cells are prostate cancer cells.

18. The method of claim 12, wherein said cancer cells are head and neck cancer cells.

19. The method of claim 18, wherein said head and neck cancer cells are squamous cell carcinoma cells.

20. A method of screening cancer cells in a sample for higher or lower aggressiveness, said method comprising visualizing the presence of SOX9 in the cytoplasm of said cells, wherein visualizing SOX9 in the cytoplasm of said cells but not in the nucleus of said cells is indicative of higher aggressiveness and the absence of SOX9 in the nucleus of said cells is indicative of lower aggressiveness, provided said cancer cells are not from a solid pseudopapillary tumor or melanoma.

21. The method of claim 20, wherein said sample is from a human patient.

22. The method of claim 20, wherein said cancer cells are breast cancer cells.

23. The method of claim 22, wherein said breast cancer cells are ductal carcinoma cells.

24. The method of claim 22, wherein said breast cancer cells are not invasive lobular carcinoma cells.

25. The method of claim 20, wherein said cancer cells are prostate cancer cells.

26. The method of claim 20, wherein said cancer cells are head and neck cancer cells.

27. The method of claim 26, wherein said head and neck cancer cells are squamous cell carcinoma cells.

28. The method of claim 20, wherein said visualization is by immunohistochemistry.

29. The method of claim 20, wherein said visualization is by immunofluorescence.
